(19) United States
(12) Patent Application Publication
(10) Pub. No.: US 2005/0085413 A1
(43) Pub. Date: Apr. 21, 2005

(54) Dimeric pharmaceutical compounds and their use

(76) Inventors: Betty Jin, Mount Waverley (AU); John N. Lambert, Blackburn (AU); Roland H. Nearn, Chelsea Heights (AU); Van T.t. Nguyen, Noble Park (AU); Simon P. Tucker, Black Rock (AU); Wen-Yang Wu, Mount Waverley (AU)

Correspondence Address: SUGHRUE MION, PLLC, 2100 PENNSYLVANIA AVENUE, N.W., SUITE 800, WASHINGTON, DC, 20037, US

(21) Appl. No.: 10/494,942

(22) PCT Filed: Nov. 8, 2002
(86) PCT No.: PCT/AU02/01525

(30) Foreign Application Priority Data
Nov. 9, 2001 (AU) PR 8796

(51) Int. Cl. C07H 17/08; C07H 15/22; A61K 31/704; A61K 38/14; C07K 9/00

(52) U.S. Cl. 514/8; 514/35; 514/28; 514/39; 530/322; 536/7.1; 536/13.2

(57) ABSTRACT

The invention relates to a compound of general formula (I):
X-L-Y (I) in which X and Y are pharmaceutically active moieties which may be the same or different; and L is a linker which is an optionally substituted saturated or unsaturated straight chain, branched and/or cyclic hydrocarbon radical having a backbone of at least 11 atoms, or a pharmaceutically acceptable derivative or salt thereof, methods for their preparation, pharmaceutical formulations containing them or their use in the prevention or treatment of a microbial infection.

This invention relates to new chemical compounds and their use in medicine. In particular the invention concerns novel dimeric compounds, methods for their preparation, pharmaceutical formulations containing them and their use as microbial agents.

### BACKGROUND OF THE INVENTION

Dimeric aminoglycosides covalently attached by a disulphide linkage having 10 carbon atoms were found to inhibit ribozyme function more effectively than their monomeric counterparts.¹ It is also known² that dimers of acetylcholinesterase (AChE) inhibitors linked by an alkylene chain of up to 10 carbon atoms are more potent than the respective monomer. However to date such compounds have only been optimised for potency. International Patent Publication No. WO 00/55149 also describes dimeric compounds which comprise two neuraminidase binding molecules, such as compound (A) shown below, attached to a common spacer or linking group of up to 100 atoms in length.

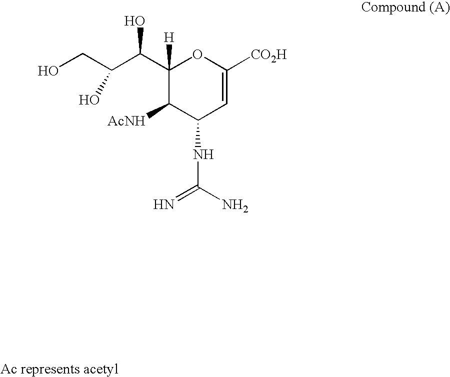

Some of the present compounds fall within the generic scope of International Patent Publication No. WO 00/55149, but are not specifically disclosed therein, and exhibit a surprisingly advantageous anti-influenza activity profile which includes an enhanced lung residency time while retaining high potency.

We have now found a linking group for pharmaceutically active dimers which leads not only to increased potency and selectivity, but also long residence time particularly at epithelial surfaces in comparison to the respective monomer. The current hypothesis is that membrane association via a hairpin-like structure is responsible for enhanced residency.

Thus the linkage of compounds via a linker group optimised for epithelial binding is expected to provide increased residence in, for example, the respiratory tract, gut, urinary tract, epithelial surfaces of the eye, skin and other sites. It is envisaged that interaction with cell membranes will also increase residence times in major organs such as the liver and central nervous system given appropriate routes of administration. Organ specific targeting groups (labile or otherwise) could also be attached to the linker to enhance the delivery process.

### SUMMARY OF THE INVENTION

In a first aspect, the present invention provides a compound of general formula (I):
X-L-Y (I)
in which
X and Y are pharmaceutically active moieties which are the same or different and selected from antiviral agents excluding inhibitors of influenza neuraminidase; aminoglycosides selected from tobramycin, kanamycin, amikacin and neomycin; antifungal agents; and antiparasitic agents; and
L is a linker which is an optionally substituted saturated or unsaturated straight chain, branched and/or cyclic hydrocarbon radical having a backbone of at least 11 atoms with the proviso that the linker does not contain one or more disulphide bonds,
or a pharmaceutically acceptable derivative or salt thereof.

The term “hydrocarbon” is used herein in its broadest sense and refers to compounds containing C and H. Examples of straight chain and branched hydrocarbons include alkyl, alkenyl or alkynyl. Examples of cyclic hydrocarbons include cycloalkyl, cycloalkenyl, heterocyclyl or aryl. It will be appreciated that the linker may include a combination of straight chain, branched and/or cyclic hydrocarbons provided that there are at least 11 atoms present in the backbone. The linker may also include one or more of N, O and S and one or more functional groups such as amide, amine, carbonyl and carboxy.

The term “alkyl” refers to straight chain or branched chain hydrocarbon groups preferably having at least 11 carbon atoms. Illustrative of such alkyl groups are dodecyl, tridecyl, tetradecyl, pentadecyl, hexadecyl, heptadecyl and octadecyl. From 12 to 16 carbon atoms are preferred.

The term “alkenyl” refers to straight chain or branched chain hydrocarbon groups preferably having at least 11 carbon atoms and having in addition one or more double bonds, of either E or Z stereochemistry where applicable. This term would include for example, farnesyl. From 12 to 16 carbon atoms are preferred.

The term “alkynyl” refers to straight chain or branched chain hydrocarbon groups having at least 11 carbon atoms and having in addition one triple bond. This term would include for example, 7-dodecynyl, 9-dodecynyl, 10-dodecynyl, 3-methyl-1-dodecyn-3-yl, 2-tridecynyl, 11-tridecynyl, 3-tetradecynyl, 7-hexadecynyl and 3-octadecynyl. From 12 to 16 carbon atoms are preferred.

The term “cycloalkyl” refers to an alicyclic group having at least 3 carbon atoms. Illustrative of such cycloalkyl groups are cyclopropyl, cyclobutyl, cyclopentyl and cyclohexyl.

The term “cycloalkenyl” refers to an alicyclic group having at least 3 carbon atoms and having in addition one or more double bonds. Illustrative of such cycloalkenyl groups are cyclopentenyl, cyclohexenyl, cycloheptenyl and cyclooctenyl.

The term “heterocyclyl” embraces saturated and partially unsaturated heteroatom-containing ring-shaped radicals, where the heteroatoms may be selected from N, S and O. Examples of saturated heterocyclyl radicals include saturated 3 to 6-membered heteromonocyclic group containing 1 to 4 nitrogen atoms for example pyrrolidinyl, imidazolidinyl, piperidino and piperazinyl; saturated 3 to 6-membered heteromonocyclic group containing 1 to 2 oxygen atoms and 1 to 3 nitrogen atoms for example morpholinyl; saturated 3 to 6-membered heteromonocyclic group containing 1 to 2 sulfur atoms and 1 to 3 nitrogen atoms for example thiazolidinyl. Examples of partially unsaturated heterocyclyl radicals include dihydrothiophene, dihydropyran, dihydrofuran and dihydrothiazole. The term “heteroaryl” embraces unsaturated heterocyclyl radicals described below.

The term “aryl” used either alone or in words such as “heteroaryl” denotes single, polynuclear, conjugated and fused residues of aromatic hydrocarbons or aromatic heterocyclic ring systems. Examples of aryl include phenyl, biphenyl, terphenyl, quaterphenyl, phenoxypenyl, naphthyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl, pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like.

In one embodiment, the linker is a straight chain or branched hydrocarbon having at least 11 C atoms, more preferably 12 to 16 C atoms, most preferably 13 or 14 C atoms.

In another embodiment, the linker may be a hydrocarbon which includes N, O, S, amide, amine, carbonyl and/or carboxy functional groups.

In a further embodiment, the linker is a hydrocarbon which includes an optionally substituted aryl such as optionally substituted phenyl, optionally substituted biphenyl or an optionally substituted heterocycle and/or N, O, S, amide, amine, carbonyl and/or carboxy functional groups.

The pharmaceutically-active moieties X and Y may be selected from synthetic or natural peptides, proteins, mono- or oligosaccharides, sugar-amino acid conjugates, sugar-peptide conjugates, toxins, drugs, pro-drugs or drug like molecules. Also included for moieties X and Y are antibodies or are antigen binding fragments of whole antibody, wherein the fragments retain the binding specificity of the whole antibody molecule. The binding fragments include, for example, Fab, F(ab′)2, and Fv fragments. Binding fragments can be obtained using conventional techniques, such as proteolytic digestion of antibody by papsin or pepsin, or through standard genetic engineering techniques that are known in the art.

The pharmaceutically-active moieties X and Y are attached to the linker L using coupling methods known in the art and compatible with the functionalities on the linker L. The term “homodimer” is used to refer to compounds of formula (I) in which X and Y are the same and the term “heterodimer” is used to refer to compounds of formula (I) in which X and Y are different. When X and Y are the same, it will be appreciated that the linker may be attached to the same or different functional groups on each moiety.

Indeed, the present invention is intended to encompass and be suitable for any pharmaceutically active moiety, especially any of the following drugs:
1. Analgesic anti-inflammatory agents such as, acetaminophen, aspirin, salicylic acid, methyl salicylate, choline salicylate, glycol salicylate, 1-menthol, camphor, mefanamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like;
2. Drugs having an action on the central nervous system, for example sedatives, hypnotics, antianxiety agents, anticholinesterase agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivocaine, bupivacaine, etidocaine, prilocaine, benzocaine, fentanyl, nicotine, galanthamine and the like;
3. Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, cloprenaline, terfenadine, chlorpheniramine, and the like;
4. Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like;
5. Steroids such as, androgenic steroids, for example, testosterone, methyltestosterone, fluoxymesterone, estrogens for example, conjugated estrogens, esterified estrogens, estropipate, 17β-estradiol, 17β-estradiol esters such as 17β-estradiol valerate, equilin, mestranol, estrone, estriol, 17β-estradiol derivatives such as 17β-ethinyl estradiol, diesthylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17α-hydroxyprogesterone, dydrogesterone, dimethisterone, ethinylestrenol, norgestrel, demegestone, promegestone, megestrol acetate, and the like;
6. Respiratory agents such as, theophylline and β₂-adrenergic agonists, for example, albuterol, terbutaline, metaproterenol, ritodrine, carbuterol, fenoterol, quinterenol, rimiterol, solmefamol, soterenol, tetroquinol, and the like;
7. Sympathomimetics such as, dopamine, norepinephrine, penylpropanolamine, pheylephrine, psuedoephedrine, amphetamine, propylhexedrine, arecoline, and the like;
8. Antimicrobial or antiinfective agents including antibacterial agents, antifungal agents, antiparasitic agents, antimycotic agents and antiviral agents, such as, those listed in the Ashgate Handbook of Anti-Infective Agents (Ed G. W. A. Milne, Ashgate Publishing, 2000), for example, tetracyclines such as oxytetracycline; penicillins such as ampicillin; cephalosporins such as cefalotin; aminoglycosides such as kanamycin, amikacin, neomycin and tobramycin; macrolides such as erythromycin, chloramphenicol, iodides, nitrofrantoin; antifungals such as clotrimazole, miconazole, chloramphenicol, nystatin, amphotericin, fradiomycin, sulfonamides, purrolnitrin, sulfacetamide, sulfamethazine, sulfadiazine, sulfamerazinre, sulfamethizole and sulfisoxazole; antivirals such as inhibitors of influenza neuraminidase and idoxuridin; clarithromycin; and other anti-infectives including nitrofurazone, and the like;
9. Antihypertensive agents such as, clonidine, α-methyldopa, reserpine, syrosingopine, rescinnamine, cinnarizine, hydrazine, prazosin, and the like;
10. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like;
11. Cardiotonics such as, digitalis, ubidecarenone, dopamine, and the like;
12. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like;
13. Vasoconstrictors such as, dihydroergotamine, dihydroergotoxine, and the like;
14. β-blockers or antiarrhythmic agents such as, timolol pindolol, propranolol, and the like;
15. Calcium antagonists and other circulatory organ agents, such as, aptopril, diltiazem, nifedipine, nicardipine, verapamil, bencyclane, ifenprodil tartarate, molsidomine, clonidine, prazosin, and the like;
16. Anti-convulsants such as, nitrazepam, meprobamate, phenytoin, and the like;
17. Agents for dizziness such as, isoprenaline, betahistine, scopolamine, and the like;
18. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like;
19. Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranquilizers such as, chlorprathixene, thiothixene, haloperidol, bromperidol, loxapine, and molindone, as well as, those agents used at lower doses in the treatment of nausea, vomiting, and the like;
20. Muscle relaxants such as, tolperisone, baclofen, dantrolene sodium, cyclobenzaprine;
21. Drugs for Parkinson's disease, spasticity, and acute muscle spasms such as levodopa, carbidopa, amantadine, apomorphine, bromocriptin, selegiline (deprenyl), trihexyphenidyl hydrochloride, benztropine mesylate, procyclidine hydrochloride, baclofen, diazepam, dantrolene, and the like;
22. Respiratory agents such as, codeine, ephedrine, isoproterenol, dextromethorphan, orciprenaline, ipratropium bromide, cromglycic acid, and the like;
23. Non-steroidal hormones or antihormones such as, corticotropin, oxytocin, vasopressin, salivary hormone, thyroid hormone, adrenal hormone, kallikrein, insulin, oxendolone, and the like;
24. Vitamins such as, vitamins A, B, C, D, E and K and derivatives thereof, calciferols, mecobalamin, and the like for dermatological use;
25. Antitumor agents such as, 5-fluorouracil and derivatives thereof, krestin, picibanil, ancitabine, cytarabine, and the like;
26. Enzymes such as, lysozyme, urokinaze, and the like;
27. Herb medicines or crude extracts such as, glycyrrhiza, aloe, Sikon (Lithospermi radix), and the like;
28. Miotics such as pilocarpine, and the like;
29. Cholinergic agonists such as, choline, acetylcholine, methacholine, carbachol, bethanechol, pilocarpine, muscarine, arecoline, and the like;
30. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eycatropine, and the like;
31. Mydriatics such as, atropine, cyclopentolate, homatropine, scopolamine, tropicamide, eucatropine, hydroxyamphetamine, and the like;
32. Psychic energizers such as, 3-(2-aminopropyl)indole, 3-(2-aminobutyl)indole, and the like;
33. Humoral agents such as, the prostaglandins, natural and synthetic, for example, PGE₁, PGE_{2α}, and PGF_{2α}, and the PGE₁ analog misoprostol.
34. Antispasmodics such as, atropine, methantheline, papaverine, cinnamedrine, methscopolamine, and the like;
35. Antidepressant drugs such as, isocarboxazid, phenelzine, tranylcypromine, imipramine, amitriptyline, trimipramine, doxepin, desipramine, nortriptyline, proptriptyline, amoxapine, maprotiline, trazodone, and the like;
36. Anti-diabetics such as, insulin, and anticancer drugs such as, tamoxifen, methotrexate, and the like;
37. Anorectic drugs such as, dextroamphetamine, methamphetamine, phenylpropanolamin, fenfluramine, diethylpropion, mazindol, phentermine, and the like;
38. Anti-allergenics such as, antazoline, methapyrilene, chlorpheniramine, pyrilamine, pheniramine, and the like;
39. Decongestants such as, phenylephrine, ephedrine, naphazoline, tetrahydrozoline, and the like;
40. Antipyretics such as, aspirin, salicylamide, and the like;
41. Antimigrane agents such as, dihydroergotamine, pizotyline, and the like;
42. Anti-malarials such as, the 4-aminoquinolines, alphaaminoquinolines, chloroquine, pyrimethamine, and the like;
43. Anti-ulcer agents such as, misoprostol, omeprazole, enprostil, allantoin, aldioxa, alcloxa, N-methylscopolamine methylsulfate, and the like;
44. Peptides such as, growth releasing factor, and the like;
45. Anti-estrogen or anti-hormone agents such as, tamoxifen or human chorionic gonadotropin, and the like.

Preferably, the pharmaceutically active moiety is an antiviral agent, for example, nucleosides, rhinovirus capsid-binding compounds, antisense oligonucleotides, peptides, inhibitors of HIVRT and inhibitors of influenza neuraminidase, for example, a compound of formula (A) defined above; an antibacterial agent such as the following aminoglycosides: 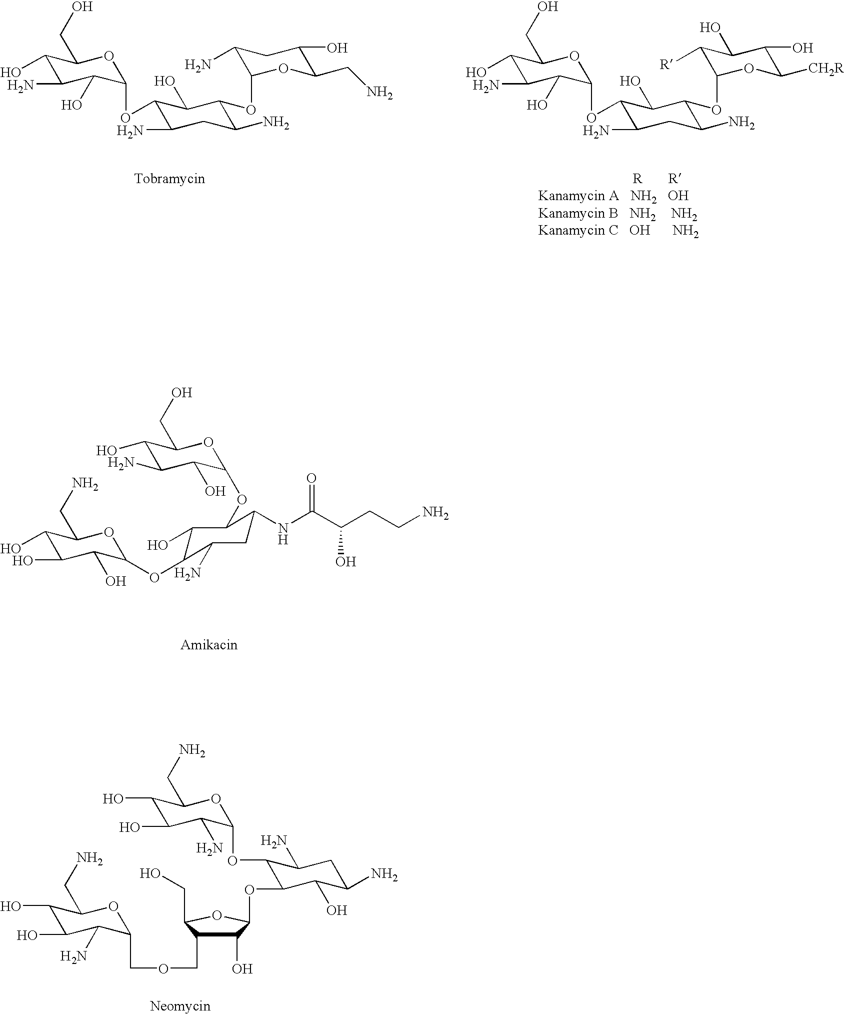
beta-lactam antibiotics, vancomycin; ciprofloxacin; antifungal agents such as amphotericin β or azoles, for example, fluconazole or ketaconazole; or antiparasitic agents such as aspartic proteinases.

It will be appreciated by those skilled in the art that the compounds of formula (I) may be modified to provide pharmaceutically acceptable derivatives thereof at any one or more of the functional groups in the compounds of formula (I). Of particular interest as such derivatives are compounds modified at the carboxyl function, hydroxyl functions or at amino groups. Thus compounds of interest include alkyl esters, such as methyl, ethyl, propyl or isopropyl esters, aryl esters, such as phenyl, benzoyl esters, and acetyl esters of the compounds of formula (I).

The term “pharmaceutically acceptable derivative” means any pharmaceutically acceptable salt, ether, ester or salt of such ester of a compound of formula (I) or any other compound which, upon administration to the recipient, is capable of providing a compound of formula (I) or a pharmaceutically active metabolite or residue thereof.

Pharmaceutically acceptable salts of the compounds of formula (I) include those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic acid, while not in themselves pharmaceutically acceptable, may be useful in the preparation of salts useful as intermediates in obtaining compounds of the invention and their pharmaceutically acceptable acid addition salts.

Salts derived from appropriate bases include alkali metal (eg. sodium), alkaline earth metal (eg. magnesium), ammonium, and NR₄⁺ (where R is C₁₋₄ alkyl) salts.

The compounds of the invention may be prepared by methods described herein. It will be apparent to those skilled in the art, that it is necessary to use protecting groups to protect one or more functional groups of the pharmaceutically active moiety during the process of attaching the pharmaceutical moiety to the linker group. See for example “Protective Groups in Organic Synthesis” by T. W. Green and P. G. M. Nuts (John Wiley & Sons, 1991).

Accordingly, the present invention also provides a method for the preparation of the compound of formula (I) as defined in any one of claims 1 to 17 which comprises the steps of:
(a) optionally protecting the pharmaceutically active moieties X and Y;
(b) reacting the optionally protected pharmaceutically active moieties X and Y with the optionally protected linker L; and
(c) if necessary, deprotecting the protected compound of formula (I).

The chemistry of the linking reaction will be determined either by the nature of reactive functional groups present in the pharmaceutical moiety or the nature of reactive groups that can be introduced to the pharmaceutical moiety using a series of chemical transformations. General methods for preparing both homodimers and heterodimers will now be described with reference to the nature of the functional group present in or introduced to the pharmaceutical moiety. It will be appreciated that the invention is not limited to such methods.

Homodimers (X-L-Y in which X=Y)

Each of the following methods can be carried out using either a pure linker of type T-L-Q or a mixture of linkers that can be described by T-L₁-Q, T-L₂-Q, T-L₃-Q, T-L₄-Q, T-L₅-Q. In the latter case, a ‘library’ of dimers would be generated.

Pharmaceutical Moieties Bearing Amines or to Which Amines Can Be Readily Introduced

1. Reductive amination

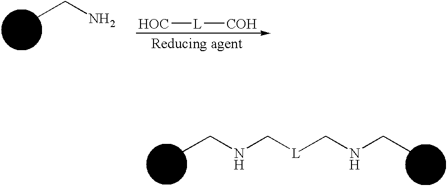

2. Amide formation

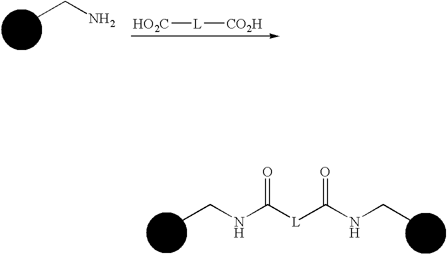

3. Urea or thiourea formation

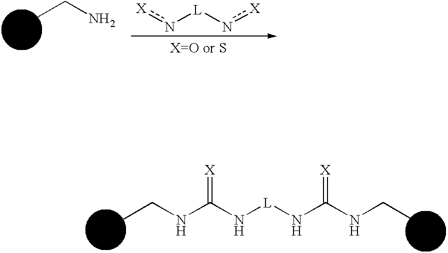

4. Imine formation

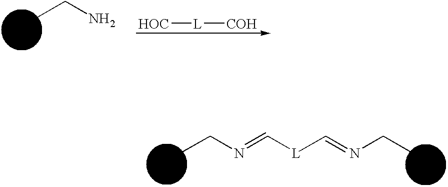

5. Alkylation by electrophiles such as halides, sulfonate derivatives and epoxides

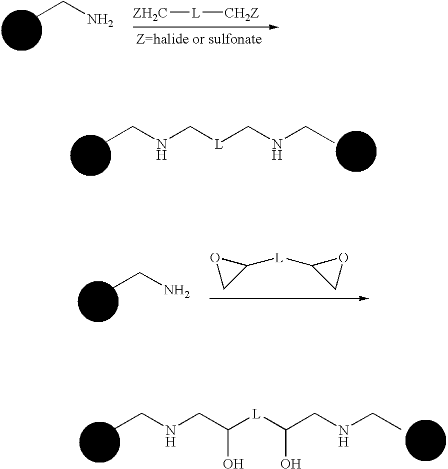

6. Conjugate addition reactions 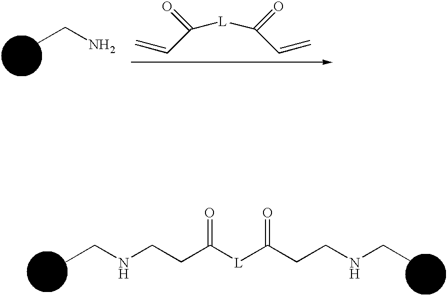
Pharmaceutical Moieties Bearing Thiols or to Which Thiols Can Be Readily Introduced

1. Thioester formation

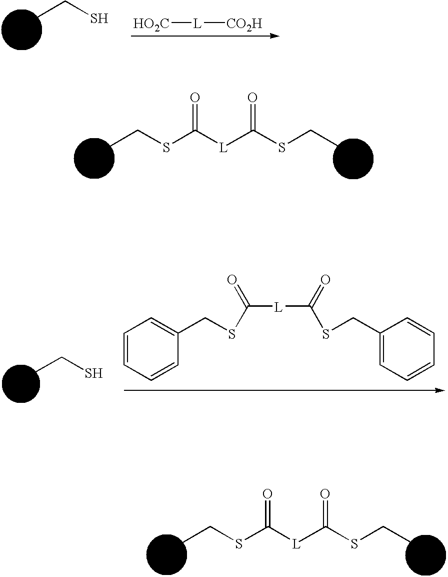

2. Thiourea or dithiourea formation

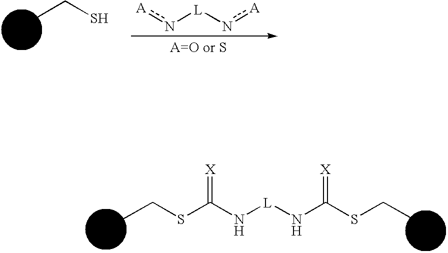

3. Alkylation by various electrophiles such as halides, sulfonate derivatives and epoxides

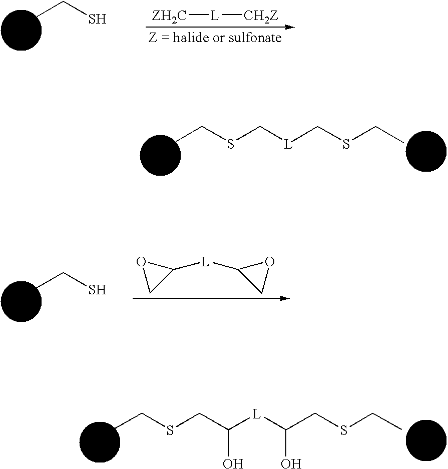

4. Conjugate addition reactions 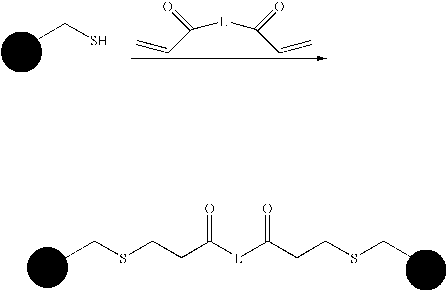
Pharmaceutical Moieties Bearing Carboxylic Acids or to Which Carboxylic Acids Can Be Readily Introduced

1. Amide formation

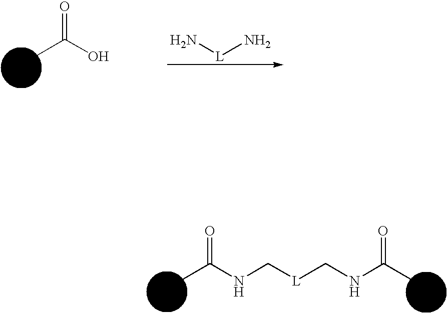

2. Amide formation (via thioesters)

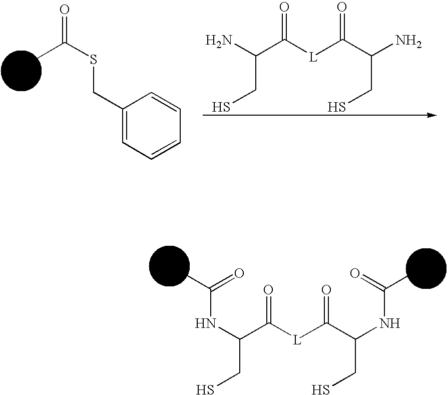

3. Ester formation 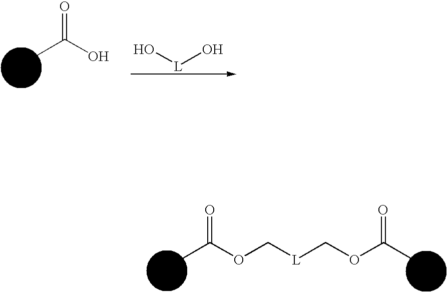
Pharmaceutical Moieties Bearing Azides or to Which Azides Can Be Readily Introduced

1. Cycloaddition 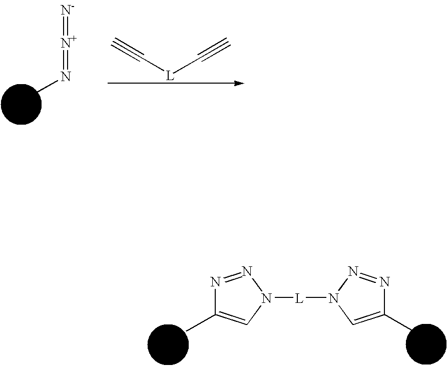
Pharmaceutical Moieties Bearing Alkynes or to Which Alkynes Can Be Readily Introduced

1. Cycloaddition

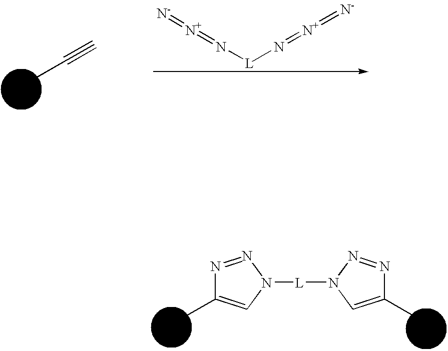

2. Organometallic coupling such as the Sonogashira reaction, the Stille reaction, the Suzuki reaction or the Negishi reaction. The Sonogashira reaction is shown below. 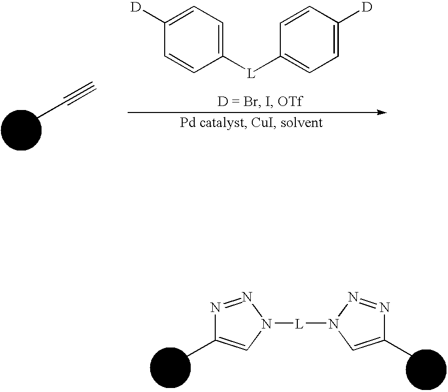
Pharmaceutical Moieties Bearing Alkenes or to Which Alkenes Can Be Readily Introduced

1. Olefin metathesis 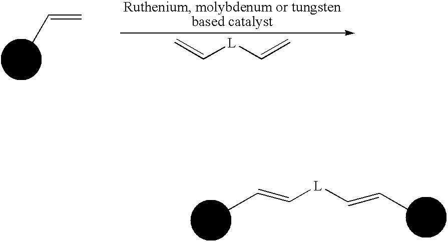
Pharmaceutical Moieties Bearing Aldehydes and Ketones or to Which Aldehydes and Ketones Can Be Readily Introduced

1. Oxime or hydrazone formation 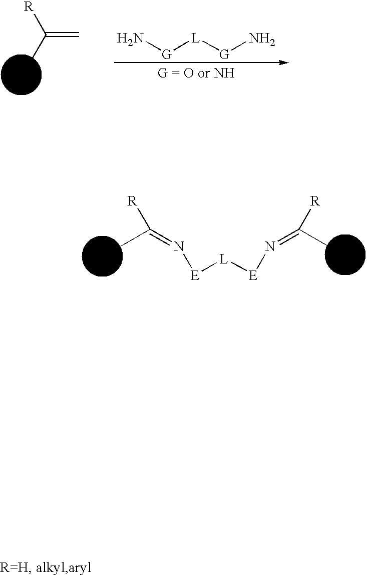
Pharmaceutical Moieties Bearing Phosphate Groups or Their Derivatives

1. Phosphate-linked dimers can be prepared as shown below. 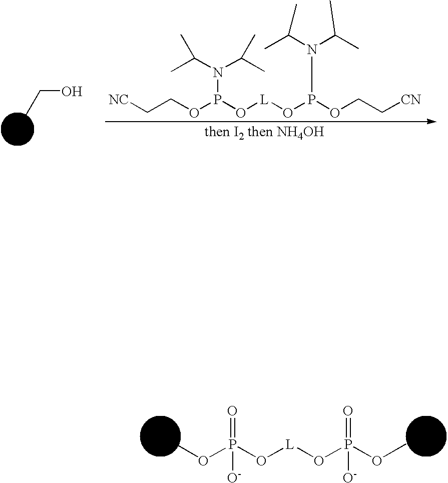
Heterodimers (X-L-Y in which X≠Y)
Synthesis of Mixtures

These may be produced using any of the above dimerisation reactions and allowing mixtures of two or more pharmaceutical moieties or their derivatives to react with a chemically compatible linker. This provides a mixture of homodimers and heterodimers that can be separated if necessary using suitable known techniques, such as, for example, high performance liquid chromatography. The composition of this mixture can be determined by the ratio of the starting materials used and the kinetics and thermodynamics of the various competing dimerisation reactions. The dimerisation reaction chosen can be selected from those provided above for homodimer formation. 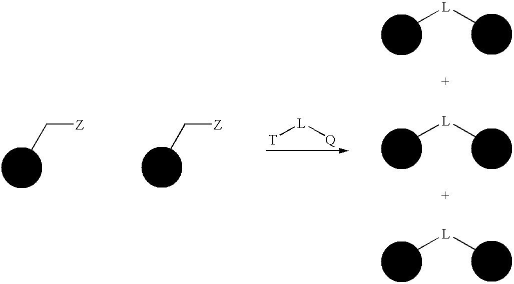
Synthesis of Heterodimers in a Controlled Manner Using Monoprotected Linkers

Heterodimers can be prepared in a controlled manner where monoprotected bifunctional linkers are sequentially modified with different pharmaceutical moieties. This is illustrated as shown below. In step 1, a carboxylic acid-equipped pharmaceutical moiety is reacted with a diamino linker that bears a Boc-protecting group at one end. After formation of an amide bond, the protecting group is removed (step 2) to expose a second amino and this is then reacted with a second carboxylic acid-equipped pharmaceutical moiety (step 3).

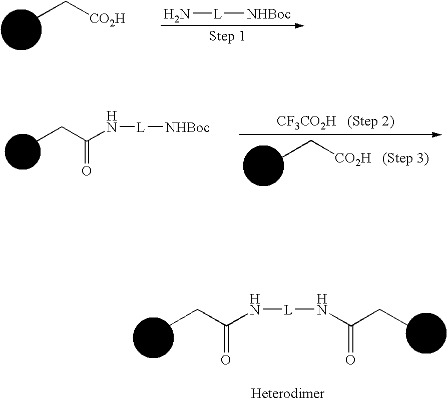

There are many variations on this method possible but those skilled in the art would be able to devise other heterodimerisation reactions by making selections from the list of reactions described above for homodimer formation.

Synthesis of Heterodimers in a Controlled Manner Using Orthogonal Reactivity

Heterodimers are preferably prepared in a controlled manner where unsymmetrical linkers equipped with orthogonally reactive groups are sequentially modified with different pharmaceutical moieties. This is best illustrated by way of an example as shown below. In step 1, an azide-equipped pharmaceutical moiety is reacted with a linker that bears a terminal alkyne and a hydroxylamine group. The azide reacts selectively with the alkyne in a cycloaddition reaction to form a triazine that may or may not need purification before the next step. In step 2, a ketone or aldehyde-bearing pharmaceutical moiety is condensed onto the hydroxylamine formed in step 1 to form an oxime.

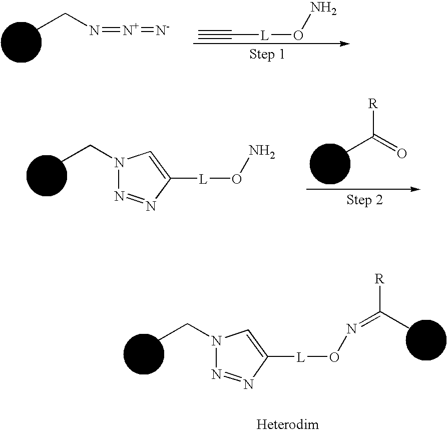

There are many variations on this method possible but those skilled in the art would be able to devise other heterodimerisation reactions by making selections from the list of reactions described above for homodimer formation. Other reactions where orthogonal reactivity is used in a similar way are shown below.

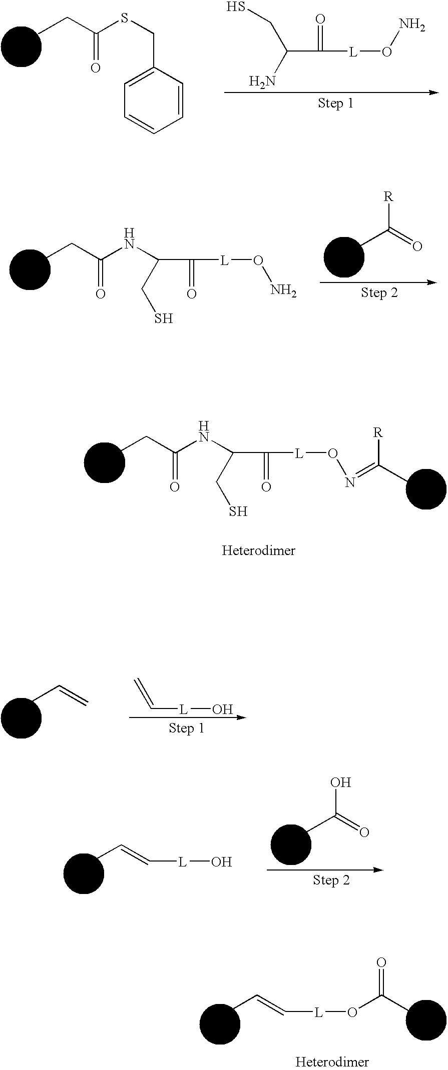

When the pharmaceutical moiety is the preferred antimicrobial agent, then dimerisation is preferably carried out with the primary goal of increasing residence time but this may also be accompanied by a increase in potency or therapeutic index. The choice of position at which dimerisation should be carried out should be guided by knowledge of how the antimicrobial agent interacts with its target or based upon known structure-activity relationships.

Antiviral Agents

Inhibitors of influenza neuraminidase can be dimerised in a number of ways to produce long residence time dimers. One example is shown below. 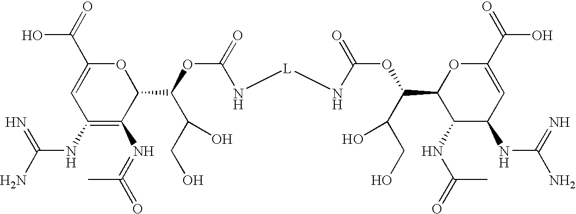
Dimers of nucleoside-based and nucleoside analogue dimers, such as, Ribavirin can be prepared as shown below. 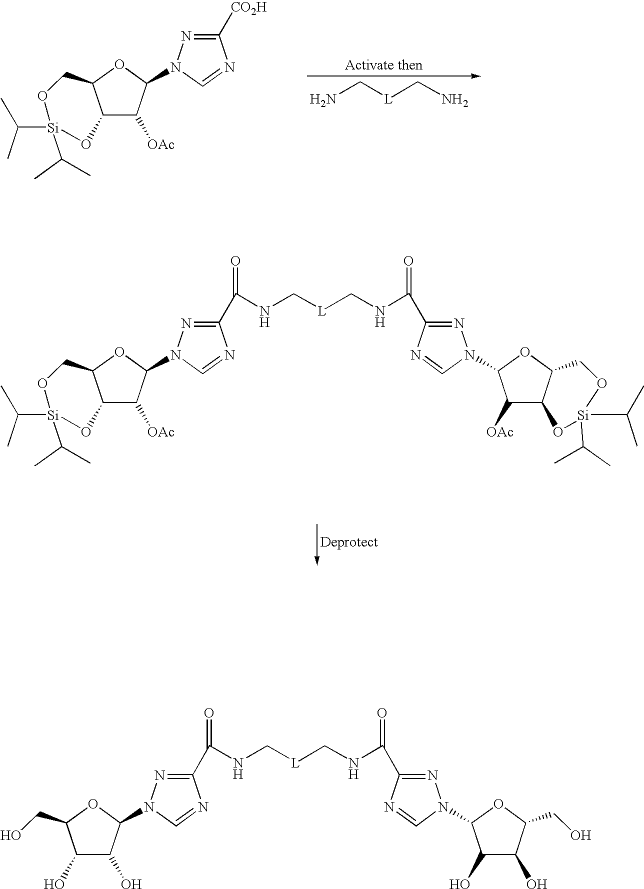
Dimers of rhinovirus capsid-binding compounds can also been prepared. 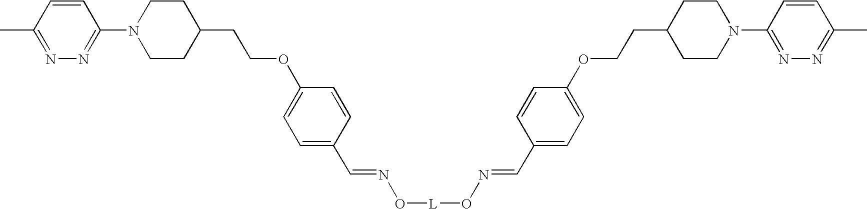
Heterodimers consisting of an antisense oligonucleotide and, for example, a peptide can be prepared as shown below (Cebon, B. et al. Aust. J. Chem., 2000, 53, 333). 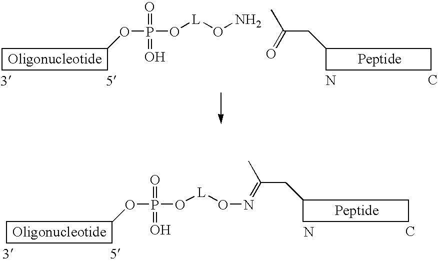
Other heterodimers based on inhibitors of HIVRT can be prepared. 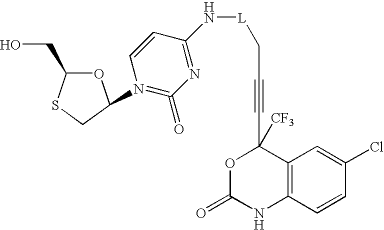
Antibacterial Agents

Dimers of beta-lactam antibiotics, for example, a dimer of Penicillin N may be prepared by activation of protected Penicillin N, reaction with a suitable diamine and deprotection as shown below.

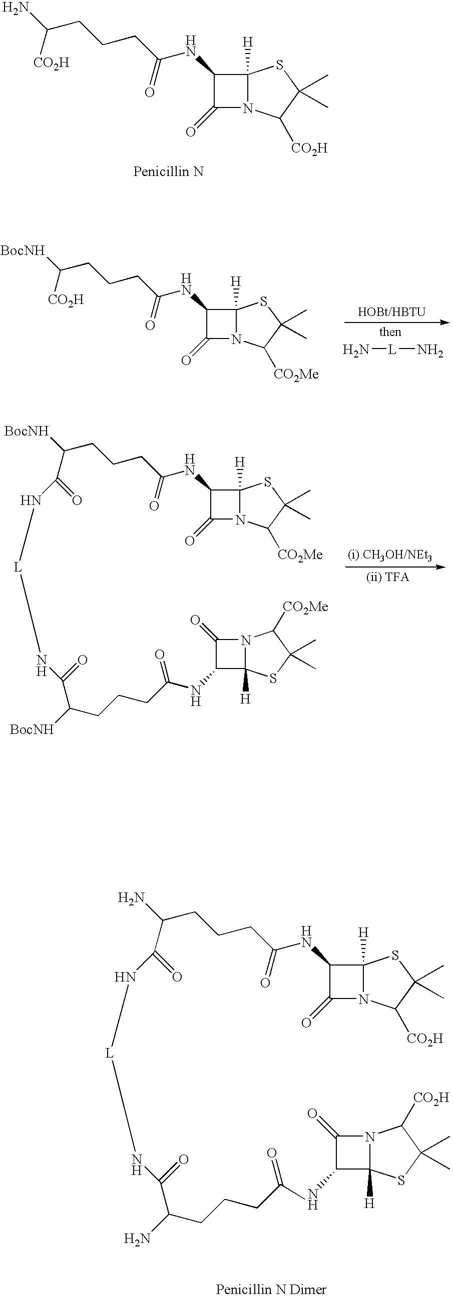

Dimers of ciprofloxacin can be prepared by reaction with a 1,ω-dialkylhalide, reductive amination with a dialdehyde or reaction with a diisocyanate as shown below.

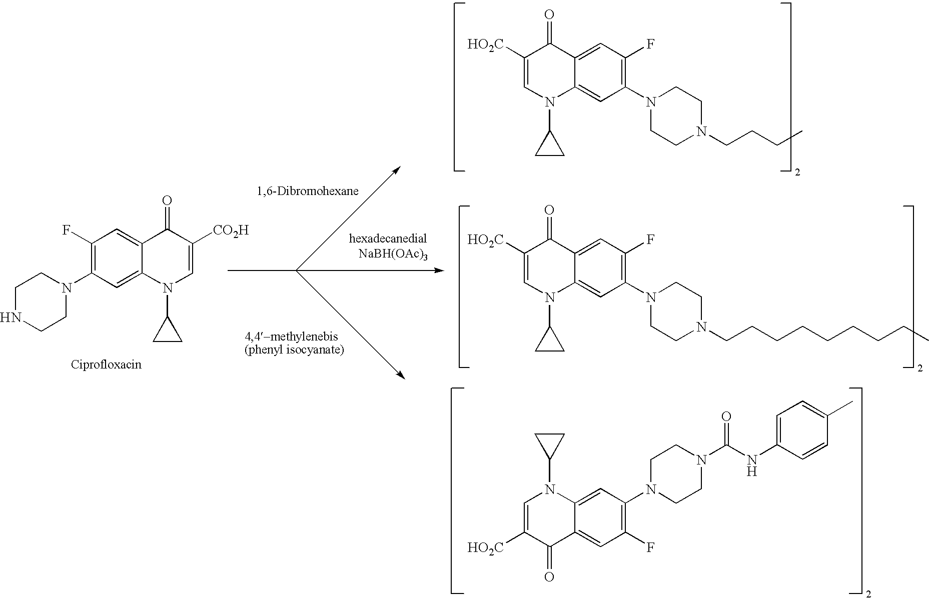

Penicillin N-aminoglycoside heterodimers may be prepared as shown below.

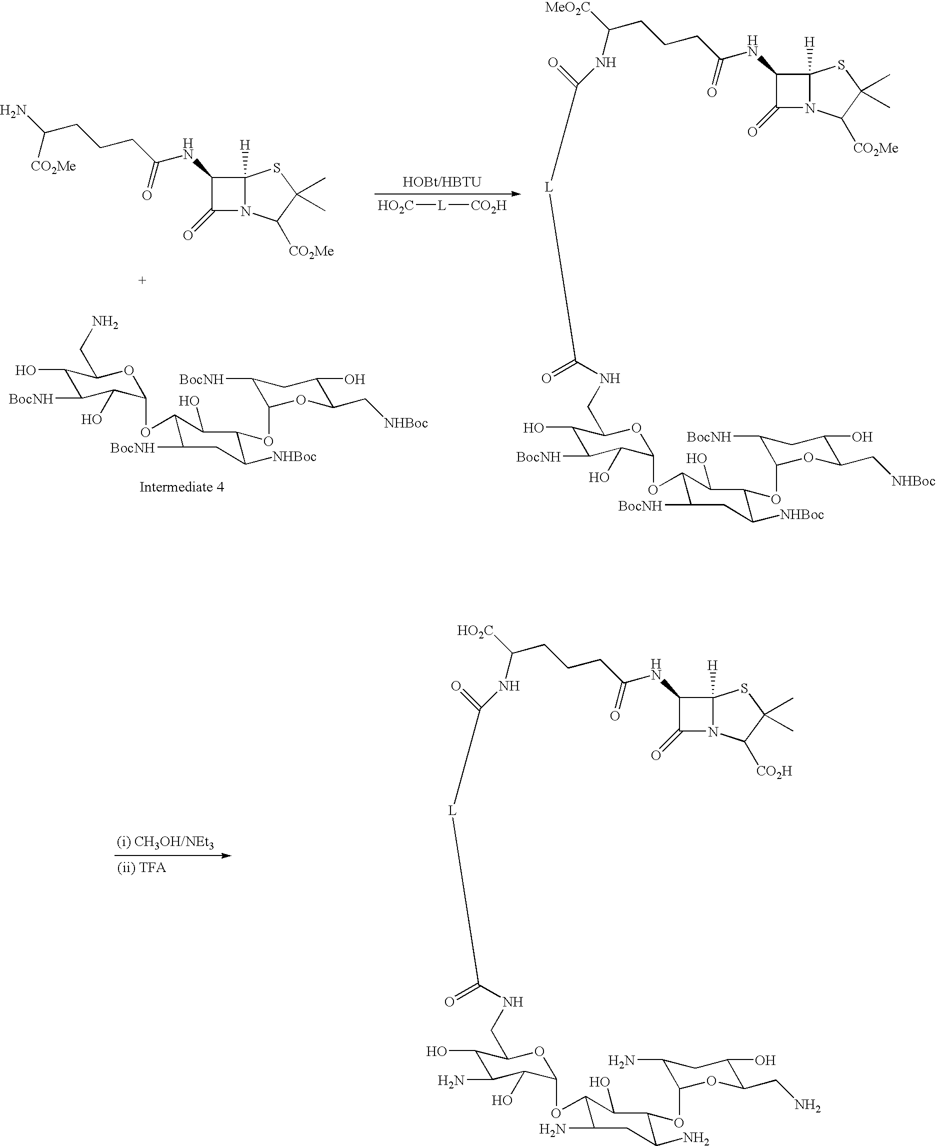

Other aminoglycoside heterodimers have been prepared using difulfide exchange reactions (Michael, K. et al., Bioorg. Med. Chem., 1999, 7, 1361).

Vancomycin homodimers may be prepared as shown below. 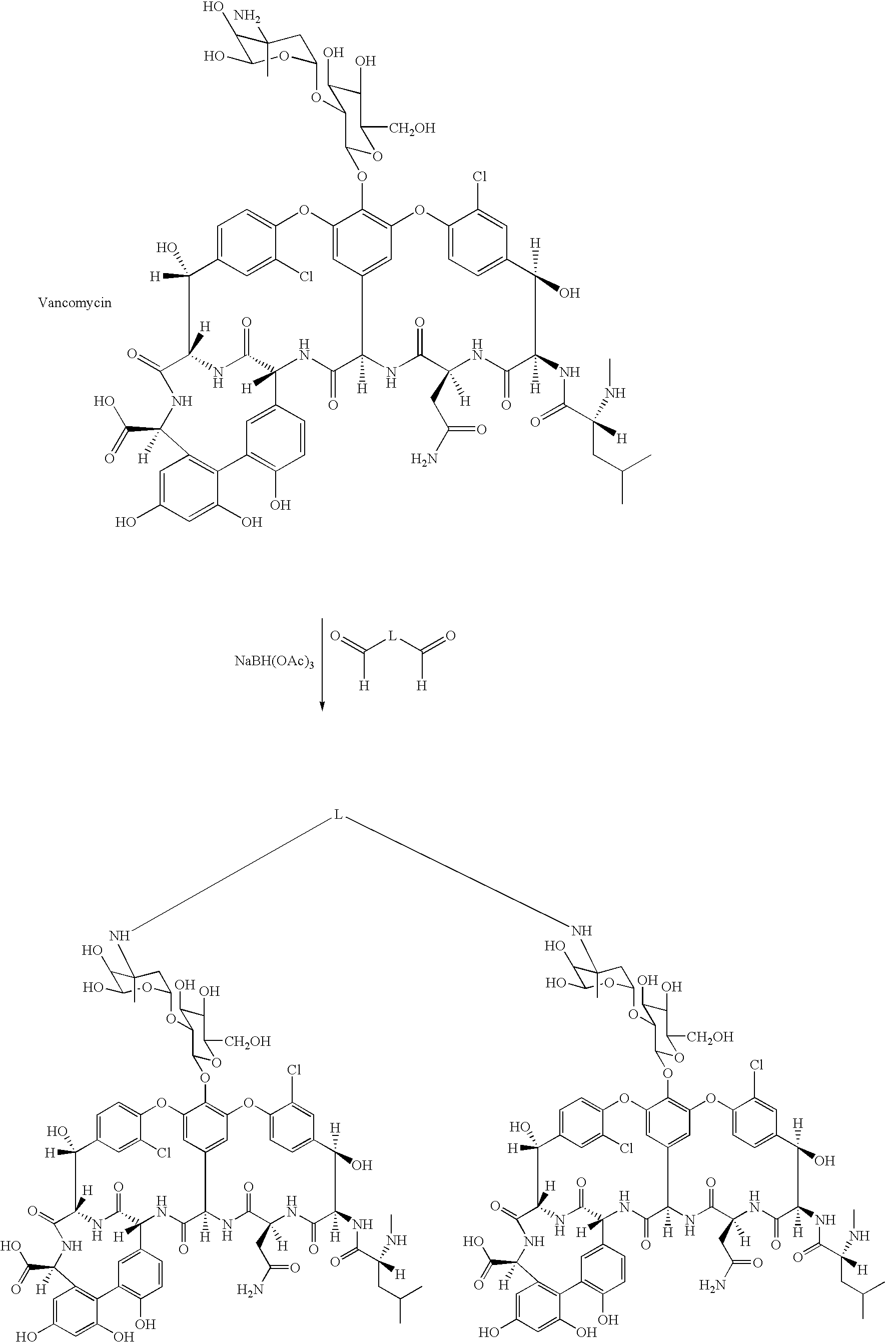
Antifungal Agents

Inhibitors of fungal cytochrome P450 14α-sterol demethylases, such as, azoles, for example, fluconazole dimers can be prepared as shown below. It has been shown that the hydroxyl group of fluconazole plays no role in receptor binding, this hydroxyl group could be used as a site through which dimers could be formed. 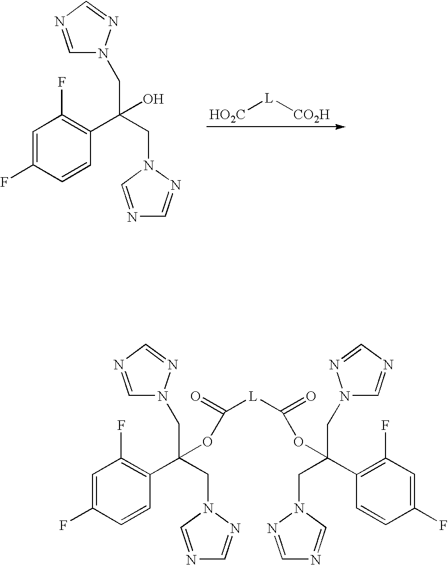
Heterodimers of azole antifungals can also be prepared, for example, fluconazole-ketoconazole heterodimers as shown below. 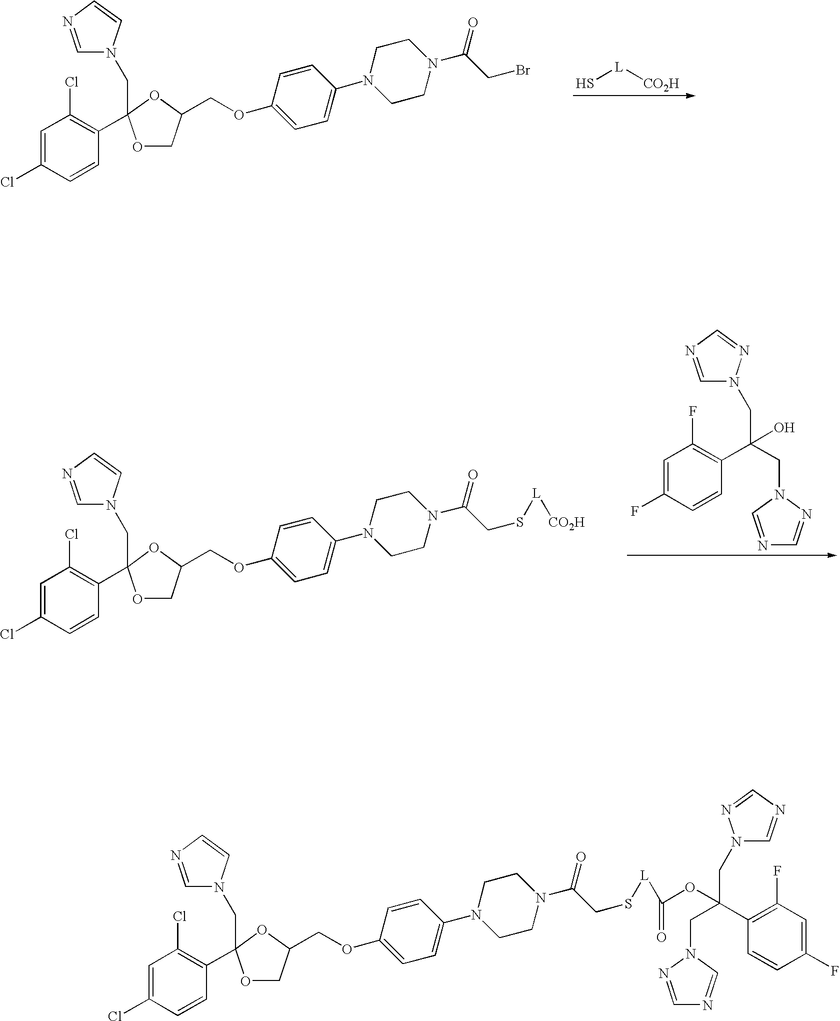
Antifungal dimers with a different mechanism of action, for example amphotericin B, can also be prepared as shown below. 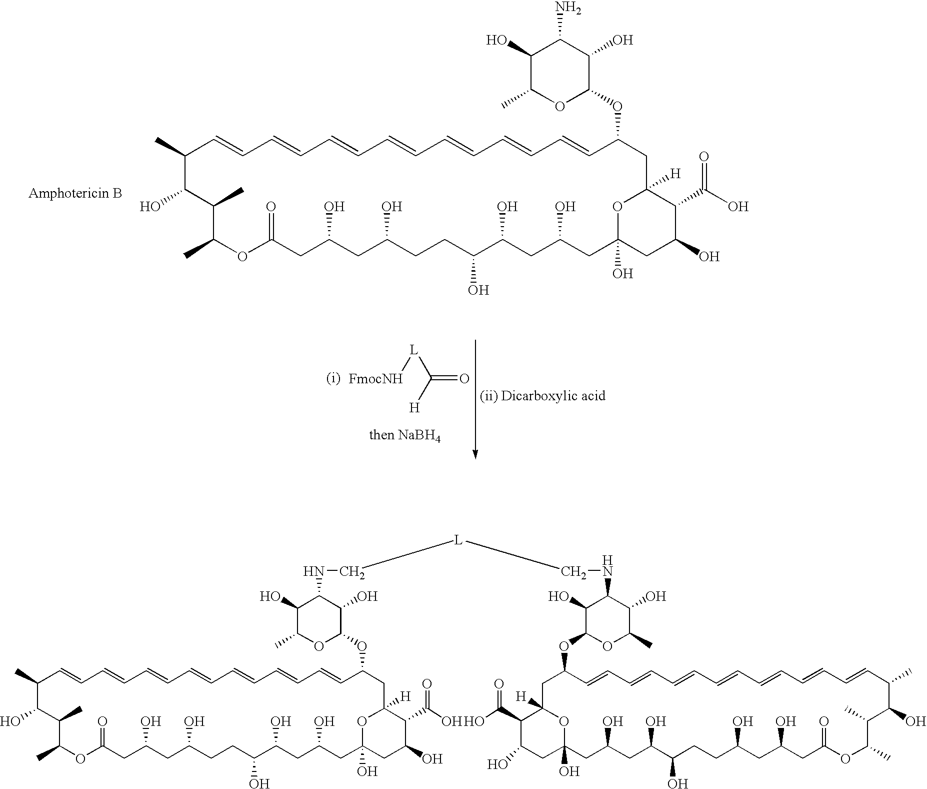
Antiparasitic Agents

Aspartic proteinases implicated in the degradation of hemoglobin by Plasmodium falciparum, for example, Plasmepsin II, is considered to be an excellent target for anti-malarial drugs. Dimeric Plasmepsin II inhibitors would be expected to exhibit long residence time in vivo and structural data (Brookhaven Protein Data Bank entry 1LEE) suggests that certain dimers of inhibitor Rs367 would be able to bind to Plasmepsin II without detriment to their enzyme inhibitory properties. The formation of one such dimer is shown below.

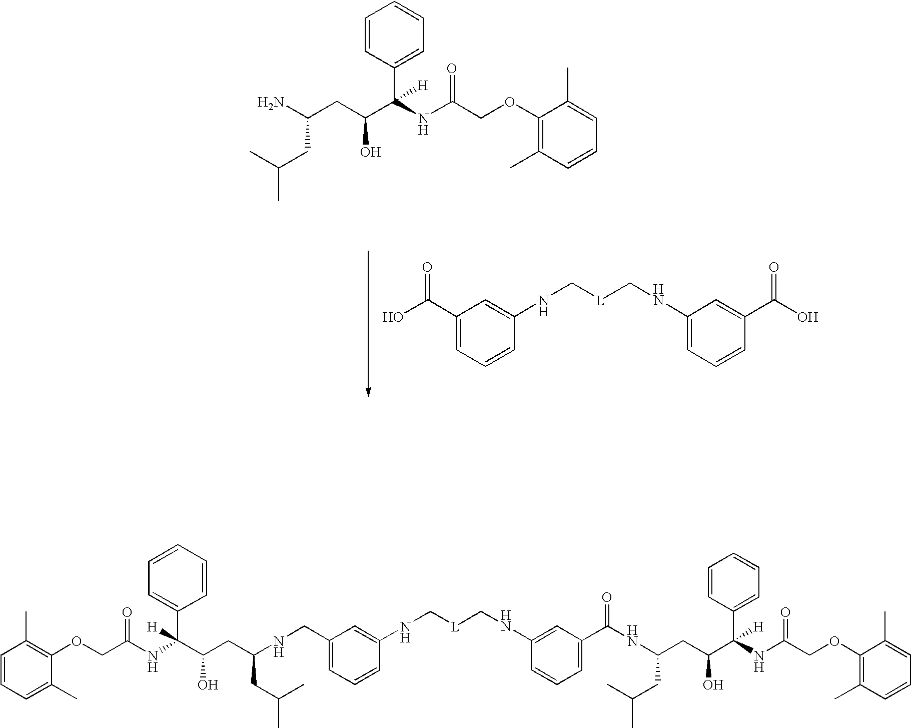

Accordingly, the present invention also extends to the preparation of the compound of formula (I) using the methods described herein.

Pharmaceutically acceptable salts of the compounds of formula (I) may be prepared according to known procedures.

For use in therapy it is preferable that the compounds of formula (I) are in crystalline form. The compounds of formula (I) depending on the nature of the pharmaceutically active moiety may possess antimicrobial activity, preferably antiviral or antibacterial activity, more preferably antiviral activity. In particular these compounds are inhibitors of viral neuraminidase of orthomyxoviruses and paramyxoviruses, for example the viral neuraminidase of influenza A and B, parainfluenza, mumps and Newcastle disease.

Thus in a second aspect the invention provides a compound of formula (I) or a pharmaceutically acceptable derivative thereof, for use as an active therapeutic agent in the treatment of a microbial infection.

In a third aspect the invention provides a method for the prevention or treatment of a microbial infection comprising the step of administration to a subject in need thereof of an effective amount of a compound of formula (I), or a pharmaceutically acceptable salt or derivative thereof.

The term “microbial infection” is used herein in its broadest sense and refers to any infection caused by a microorganism and includes viral and bacterial infections. Examples of such infectious microorganisms may be found in a number of well known texts such as ‘Medical Microbiology’ (Greenwood, D., Slack, R., Peutherer, J., Churchill Livingstone Press, 2002); ‘Mims’ Pathogenesis of Infectious Disease’ (Mims, C., Nash, A., Stephen, J., Academic Press, 2000); “Fields” Virology. (Fields, B. N., Knipe, D. M., Howley, P. M., Lippincott Williams and Wilkins, 2001).

The term “microorganism” includes any microscopic organism or taxonomically related macroscopic organism within the categories algae, bacteria, fungi, protozoa, viruses and subviral agents or the like. Although, the preferable microorganism is those found in sources described above. For example those microorganisms found in anaerobic sludge such as methanogens, eubacteria or nitrifying bacteria.

Viral infections include, but are not limited to those caused by Adenovirus, Lassa fever virus (Arenavirus), Astrovirus, Hantavirus, Rift Valley Fever virus (Phlebovirus), Calicivirus, Ebola virus, Marburg Virus, Japanese encephalitis virus, Dengue virus, Yellow fever virus, Hepatitis C virus, Hepatitis G virus, Hepatitis B virus, Hepatitis D virus, Herpes simplex virus 1, Herpes simplex virus 2, Cytomegalovirus, Epstein Barr virus, Varicella Zoster Virus, Human Herpesvirus 7, Human Herpesvirus 8, Influenza virus, Parainfluenza virus, Rubella virus, Mumps virus, Morbillivirus, Measles virus, Respiratory Syncytial virus, Papillomaviruses, JC virus (Polyomavirus), BK virus (Polyomavirus), Parvovirus, Coxsackie virus (A and B), Hepatitis A virus, Polioviruses, Rhinoviruses, Reovirus, Rabies Virus (Lyssavirus), Human Immunodeficiency virus 1 and 2 and Human T-cell Leukemia virus.

Examples of viral infections include Adenovirus acute respiratory disease, Lassa fever, Astrovirus enteritis, Hantavirus pulmonary syndrome, Rift valley fever, Hepatitis E, diarrhoea, Ebola hemorrhagic fever, Marburg hemorrhagic fever, Japanese encephalitis, Dengue fever, Yellow fever, Hepatitis C, Hepatitis G, Hepatitis B, Hepatitis D, Cold sores, Genital sores, Cytomegalovirus infection, Mononucleosis, Chicken Pox, Shingles, Human Herpesvirus infection 7, Kaposi Sarcoma, Influenza, Brochiolitis, German measles, Mumps, Measles (rubeola), Measles, Brochiolitis, Papillomas (Warts), cervical cancer, Progressive multifocal leukoencephalopathy, Kidney disease, Erythema infectiosum, Viral myocarditis, meninigitis, entertitis, Hepititis, Poliomyelitis, Cold, Diarrhoea, Rabies, AIDS and Leukemia.

Bacterial infections include, but are not limited to, infections caused by Gram Positive Bacteria including Bacillus cereus, Bacillus anthracis, Clostridium botulinum, Clostridium difficile, Clostridium tetani, Clostridium perfringens, Corynebacteria diphtheriae, Enterococcus (Streptococcus D), Listeria monocytogenes, Pneumoccoccal infections (Streptococcus pneumoniae), Staphylococcal infections and Streptococcal infections; Gram Negative Bacteria including Bacteroides, Bordetella pertussis, Brucella, Campylobacter infections, enterohaemorrhagic Escherichia coli (EHEC/E. coli 0157: H7) enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonnorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulare, Myobacterium johnei, Mycobacterium leprae, atypical bacteria, Chlamydia, Mycoplasma, Rickettsia, Spirochetes, Treponema pallidum, Borrelia recurrentis, Borrelia burgdorfii and Leptospira icterohemorrhagiae; or other miscellaneous bacteria, including Actinomyces and Nocardia.

Preferably, the bacterial infection is a Gram Negative or Gram Positive infection such as infections associated with the respiratory tract (e.g. pneumonia associated with Klebsiella, mycobacterium species including tuberculosis and pseudomonas aeruginosa), urinary tract and systemic infections caused by enteric bacteria, GI tract diseases such as Shigella dysentery and plague.

Fungal infections include, but are not limited to, infections caused by Alternaria alternata, Aspergillus flavus, Aspergillus fumigatus, Aspergillus nidulans, Aspergillus niger, Aspergillus versicolor, Blastomyces dermatiditis, Candida albicans, Candida dubliensis, Candida krusei, Candida parapsilosis, Candida tropicalis, Candida glabrata, Coccidioides immitis, Cryptococcus neoformans, Epidermophyton floccosum, Histoplasma capsulatum, Malassezia furfur, Microsporum canis, Mucor spp., Paracoccidioides brasiliensis, Penicillium marneffei, Pityrosporum ovale, Pneumocystis carinii, Sporothrix schenkii, Trichophyton rubrum, Trichophyton interdigitale, Trichosporon beigelii and Rhodotorula spp.

Yeast infections include, but are not limited to, infections caused by Brettanomyces clausenii, Brettanomyces custerii, Brettanomyces anomalous, Brettanomyces naardenensis, Candida himilis, Candida intermedia, Candida saki, Candida solani, Candida tropicalis, Candida versatilis, Candida bechii, Candida famata, Candida lipolytica, Candida stellata, Candida vini, Debaromyces hansenii, Dekkera intermedia, Dekkera bruxellensis, Geotrichium sandidum, Hansenula fabiani, Hanseniaspora uvarum, Hansenula anomala, Hanseniaspora guillermondii Hanseniaspora vinae, Kluyveromyces lactis, Kloekera apiculata, Kluveromyces marxianus, Kluyveromyces fragilis, Metschikowia pulcherrima, Pichia guilliermodii, Pichia orientalis, Pichia fermentans, Pichia memranefaciens, Rhodotorula Saccharomyces bayanus, Saccharomyces cerevisiae, Saccharomyces dairiensis Saccharomyces exigus, Saccharomyces uinsporus, Saccharomyces uvarum, Saccharomyces oleaginosus, Saccharomyces boulardii, Saccharomycodies ludwigii, Schizosaccharomyces pombe, Torulaspora delbruekii, Torulopsis stellata, Zygoaccharomyces bailli and Zygosaccharomyces rouxii.

Protozoal infections include, but are not limited to, infections caused by Leishmania, Toxoplasma, Plasmodia, Theileria, Anaplasma, Giardia, Trichomonas, Trypanosoma, Coccidia, and Babesia. Specific examples include Trypanosoma cruzi, Eimeria tenella, Plasmodium falciparum, Plasmodium vivax or Plasmodium ovale.

Preferably the subject is an animal such as a mammal, more preferably a human, or a member of the genus Equus, for example a horse, donkey or mule. Most preferably the mammal is a human.

In a fourth aspect the invention provides use of a compound of the invention for the manufacture of a medicament for the treatment of a microbial infection.

As used herein, the term “effective amount” is meant an amount of the compound of formula I effective to preventing or treating a microbial infection in order to yield a desired therapeutic response. For example, to overcome or alleviate the effects of a microbial infection.

The term “therapeutically-effective amount” means an amount of the compound of formula I to yield a desired therapeutic response. For example, treating or preventing a microbial infection.

The specific “therapeutically-effective amount” will, obviously, vary with such factors as the particular microbial infection being treated, the physical condition of the subject, the type of animal being treated, the duration of the treatment, the nature of concurrent therapy (if any), and the specific formulation employed and the structure of the compound or its derivatives.

Generally, the terms “treating”, “treatment” and the like are used herein to mean affecting a subject, tissue or cell to obtain a desired pharmacologic and/or physiologic effect. The effect may be prophylactic in terms of completely or partially preventing a microbial infection or sign or symptom thereof, and/or may be therapeutic in terms of a partial or complete cure of a microbial infection. “Treating” as used herein covers any treatment of, or prevention of a microbial infection in a vertebrate, a mammal, particularly a human, and includes: (a) preventing the microbial infection from occurring in a subject that may be predisposed to the viral or bacterial infection, but has not yet been diagnosed with the microbial infection; (b) inhibiting the microbial infection, i.e., arresting its development; or (c) relieving or ameliorating the effects, i.e., cause regression of the symptoms of the microbial infection.

The compounds of the invention may also be used in diagnostic methods, in particular methods for the detection of microbial infections such as the influenza virus. For use in such methods it may be advantageous to link a compound of the invention to a label, such as a radioactive, fluorescent or chemiluminescent label.

Methods of diagnosis for which the compounds of the invention are suitable are described, for example, in our earlier applications PCT/AU97/00109 and PCT/AU97/00771.

In a fifth aspect the invention provides a method for the detection of a microbial infection which comprises the step of contacting the compound of the invention with a sample suspected of containing the microorganism.

It will be further appreciated that the amount of a compound of the invention required for use in treatment will vary not only with the particular compound selected but also with the route of administration, the nature of the condition being treated, and the age and condition of the subject, and will ultimately be at the discretion of the attendant physician or veterinarian. In general however, a suitable dose will be in the range of from about 0.001 to 100 mg/kg of bodyweight per day, preferably in the range of 0.001 to 1 mg/kg/day, most preferably in the range of 0.002 to 0.1 mg/kg/day.

Treatment is preferably commenced before or at the time of infection and continued until microorganism is no longer present. However the compounds are also effective when given post-infection, for example after the appearance of established symptoms.

Suitably treatment is given on one or two occasions, preferably only once only for treatment, and preferably once per week for prophylaxis.

The compound is conveniently administered in unit dosage form, for example containing 1 to 100 mg, more conveniently 0.1 to 10 mg, most conveniently 0.1 to 5 mg of active ingredient per unit dosage form.

While it is possible that, for use in therapy, a compound of the invention may be administered as the raw chemical, it is preferable to present the active ingredient as a pharmaceutical formulation.

Thus in a sixth aspect the invention provides a pharmaceutical formulation comprising a compound of formula (I) or a pharmaceutically acceptable salt or derivative thereof, together with one or more pharmaceutically acceptable carriers therefor and, optionally, other therapeutic and/or prophylactic ingredients. The carrier(s) must be “acceptable” in the sense of being compatible with the other ingredients of the formulation and not being deleterious to the recipient thereof.

The compounds of the invention may also be used in combination with other therapeutic and/or prophylactic agents, for example other anti-infective agents. In particular the compounds of the invention may be employed with other antiviral agents. The invention thus provides in a seventh aspect a combination comprising a compound of formula (I) or a pharmaceutically acceptable salt or derivative thereof together with another therapeutically and/or prophylactically active agent, in particular an antimicrobial agent.

The combinations referred to above may conveniently be presented for use in the form of a pharmaceutical formulation and thus such formulations comprising a combination as defined above together with a pharmaceutically acceptable carrier therefor comprise a further aspect of the invention.

Suitable therapeutic and/or prophylactic agents for use in such combinations include other anitmicrobial agents, in particular anti-bacterial and anti-viral agents such as those used to treat respiratory infections. For example, other compounds or vaccines effective against influenza viruses, such as the sialic acid analogues referred to above, e.g. zanamivir, oseltamivir, amantadine, rimantadine and ribavirin and FluVax, may be included in such combinations.

The individual components of such combinations may be administered either separately, sequentially or simultaneously in separate or combined pharmaceutical formulations.

When the compounds of the invention are used with a second therapeutic and/or prophylactic agent active against the same virus, the dose of each compound may either be the same as or different from that employed when each compound is used alone. Appropriate doses will be readily appreciated by those skilled in the art.

Pharmaceutical formulations include those suitable for oral, rectal, nasal, topical (including buccal and sub-lingual), vaginal or parenteral (including intramuscular, sub-cutaneous and intravenous) administration, or those in a form suitable for administration to the respiratory tract (including the nasal passages) for example by inhalation or insufflation. The formulations may, where appropriate, be conveniently presented in discrete dosage units, and may be prepared by any of the methods well known in the art of pharmacy. These methods include the step of bringing into association the active compound with liquid carriers or finely divided solid carriers or both, and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may for example be in the form of aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles, which may include edible oils, or preservatives.

The compounds according to the invention may also be formulated for parenteral administration by injection, for example bolus injection, or continuous infusion, and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulating agents such as suspending, stabilising and/or dispersing agents. Alternatively, the active ingredient may be in powder form, obtained by aseptic isolation of sterile solid or by lyophilisation from solution, for constitution with a suitable vehicle, eg. sterile, pyrogen-free water, before use.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base, and will in general also contain one or more emulsifying agents, stabilising agents, dispersing agents, suspending agents, thickening agents, or colouring agents.

Formulations suitable for topical administration in the mouth include lozenges comprising active ingredient in a flavoured base, usually sucrose and gum acacia or gum tragacanth; pastilles comprising the active ingredient in an inert base such as gelatin or sucrose and gum acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping moulds.

Formulations suitable for vaginal administration may be presented as pessaries, tampons, creams, gels, pastes, foams or sprays containing in addition to the active ingredient such carriers as are known in the art to be appropriate.

For administration to the respiratory tract, including intranasal administration, the neuraminidase inhibitors may be administered by any of the methods and formulations employed in the art for administration to the respiratory tract.

Thus in general the compounds may be administered in the form of a solution or a suspension or as a dry powder.

Solutions and suspensions will generally be aqueous, for example prepared from water alone (for example sterile or pyrogen-free water) or water and a physiologically acceptable co-solvent (for example ethanol, propylene glycol or polyethylene glycols such as PEG 400).

Such solutions or suspensions may additionally contain other excipients for example preservatives (such as benzalkonium chloride), solubilising agents/surfactants such as polysorbates (eg. Tween 80, Span 80, benzalkonium chloride), buffering agents, isotonicity-adjusting agents (for example sodium chloride), absorption enhancers and viscosity enhancers. Suspensions may additionally contain suspending agents (for example microcrystalline cellulose, carboxymethyl cellulose sodium).

Solutions or suspensions are applied directly to the nasal cavity by conventional means, for example with a dropper, pipette or spray. The formulations may be provided in single or multidose form. In the latter case a means of dose metering is desirably provided. In the case of a dropper or pipette this may be achieved by the patient administering an appropriate, predetermined volume of the solution or suspension. In the case of a spray this may be achieved for example by means of a metering atomising spray pump.

Administration to the respiratory tract may also be achieved by means of an aerosol formulation in which the compound is provided in a pressurised pack with a suitable propellant, such as a chlorofluorocarbon (CFC), for example dichlorodifluoromethane, trichlorofluoromethane or dichlorotetrafluoroethane, carbon dioxide or other suitable gas. The aerosol may conveniently also contain a surfactant such as lecithin. The dose of drug may be controlled by provision of a metered valve.

Alternatively the compounds may be provided in the form of a dry powder, for example a powder mix of the compound in a suitable powder base such as lactose, starch, starch derivatives such as hydroxypropylmethyl cellulose and polyvinylpyrrolidine (PVP). Conveniently the powder carrier will form a gel in the nasal cavity. The powder composition may be presented in unit dose form, for example in capsules or cartridges of eg. gelatin, or blister packs from which the powder may be administered by means of an inhaler.

In formulations intended for administration to the respiratory tract, including intranasal formulations, the compound will generally have a small particle size, for example of the order of 5 microns or less. Such a particle size may be obtained by means known in the art, for example by micronisation.

When desired, formulations adapted to give sustained release of the active ingredient may be employed.

Preferably the compounds of the invention are administered to the respiratory tract by inhalation, insufflation or intranasal administration, or a combination thereof.

“Relenza” is administered by oral inhalation as a free-flow powder via a “Diskhaler” (trade mark of Glaxo Wellcome plc). A similar formulation would be suitable for the present invention.

Thus, according to an eighth aspect of the present invention there is provided an inhaler which contains a formulation as defined above.

It will be appreciated that the inhaler may also be in the form of a meter dose aerosol inhaler.

For the purposes of this specification it will be clearly understood that the word “comprising” means “including but not limited to”, and that the word “comprises” has a corresponding meaning.

All publications, including but not limited to patents and patent applications, cited in this specification are herein incorporated by reference as if each individual publication were specifically and individually indicated to be incorporated by reference herein as though fully set forth.

### DETAILED DESCRIPTION OF THE INVENTION

The invention will now be described in detail by way of reference only to the following non-limiting examples.

Antiviral Agents

### EXAMPLES 1 TO 8

Machine Methods

Method A (LC/MS)

Micromass Platform II mass spectrometer operating in positive ion electrospray mode, mass range 100-1000 amu.
Column: 3.3 cm×4.6 mm ID, 3 μm ABZ+PLUS
Flow Rate: 3 ml/min
Injection Volume: 5 μl
Solvent A: 95% acetonitrile+0.05% formic acid
Solvent B: 0.1% formic acid+10 mMolar ammonium acetate
Gradient: 0-100% A/5 min, 100-0% B/5 min
   Method B

The prep column used was a Supelcosil ABZ plus (10 cm×2.12 cm).
UV wavelength: 230 nm
Flow: 4 ml/min
Solvent A: acetonitrile+0.05% TFA
Solvent B: water+0.1% TFA
Gradient: 20-40% A/20 min, 40% A/20 min, 40-100% A/0.3 min, 100% A/15 min, 100-20% A/3 min
   Abbreviations

TFA trifluoroacetic acid

DMAP 4-dimethylaminopyridine

SPE solid phase extraction
Preparation of Intermediate 1

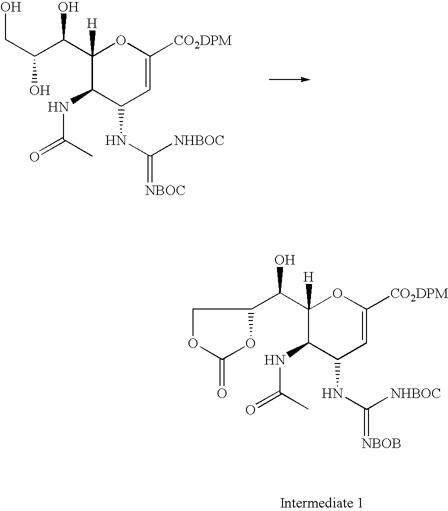

Benzhydryl (2R,3R,4S)-3-(acetylamino)-4-({(E)-[(tert-butoxycarbonyl)amino][(tert-butoxycarbonyl)imino]methyl}amino)-2-[(1R,2R)-1,2,3-trihydroxypropyl]-3,4-dihydro-2H-pyran-6-carboxylate (see J. Med. Chem. 1998, 41, 787-797) (12.38 g; 17.7 mmoles) was dissolved in dry acetonitrile (130 ml) under nitrogen at room temperature. The solution was stirred and 1,1′-carbonyldiimidazole (2.87 g; 17.7 mmoles) was added. After 16 hours LC/MS showed the presence of starting triol so further 1,1′-carbonyldiimidazole (total of 0.493 g; 3 mmoles) was added. After a few hours LC/MS showed no triol present. The solvent was evaporated and the residue flash columned on silica, eluting with 1:1 ethyl acetate/40-60 petroleum ether. Fractions containing wanted product were evaporated then taken up in dichloromethane, dried with sodium sulphate, filtered and evaporated to give Intermediate 1 (benzhydryl (2R,3R,4S)-3-(acetylamino)-4-({[(tert-butoxycarbonyl)amino][(tert-butoxycarbonyl)imino]methyl}amino)-2-{(S)-hydroxy[(4R)-2-oxo-1,3-dioxolan-4-yl]methyl}-3,4-dihydro-2H-pyran-6-carboxylate) as an off white solid (11.05 g; 86%).
Preparation of Intermediate 10

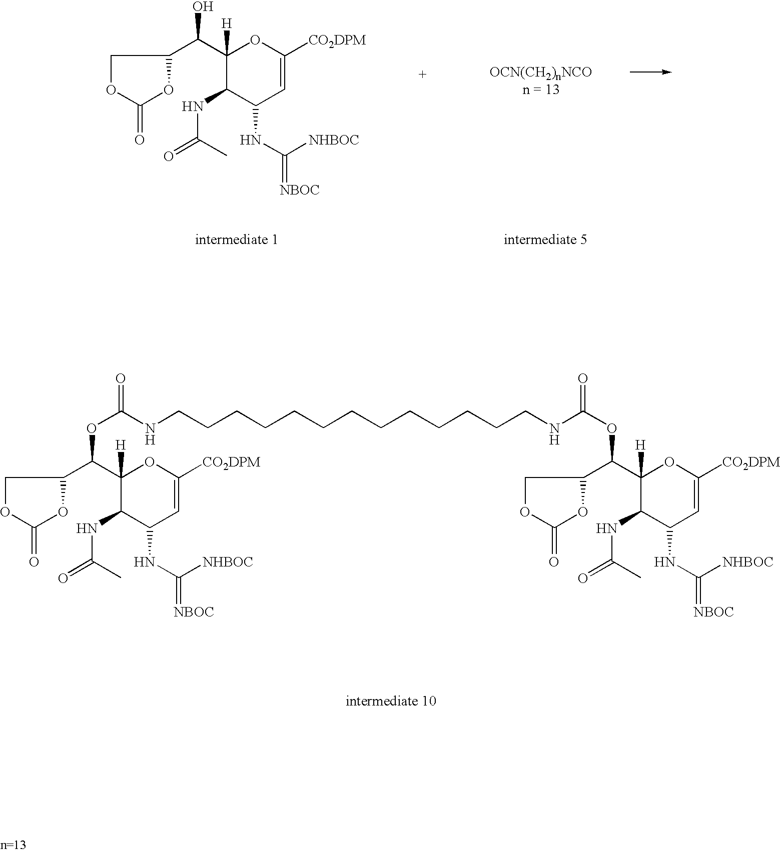

Intermediate 1 (0.4 g; 0.56 mmole) was dissolved in dry dichloromethane (0.5 ml). To this was added DMAP (20 mg) and 4 molecular sieves type 3A followed by intermediate 5 (50 mg; 0.19 mmole). The mixture was refluxed overnight then applied directly to a 10 g Si SPE cartridge eluted with diethyl ether and ethyl acetate to give intermediate 10 as a colourless glass (0.16 g, 50% yield).

LC/MS (method A) showed (M+2H⁺)/2=858; T_{RET}=4.68 min.

Similarly prepared were the following:

| n | diisocyanate | dicarbamate | (M + 2H⁺)/2 | T_{RET}(min) |
|---|---|---|---|---|
| 11 | intermediate 3 | intermediate 8 | 844 | 4.68 |
| 12 | intermediate 4 | intermediate 9 | 851 | 4.66 |
| 14 | intermediate 6 | intermediate 11 | 865 | 4.75 |

Preparation of Intermediate 15

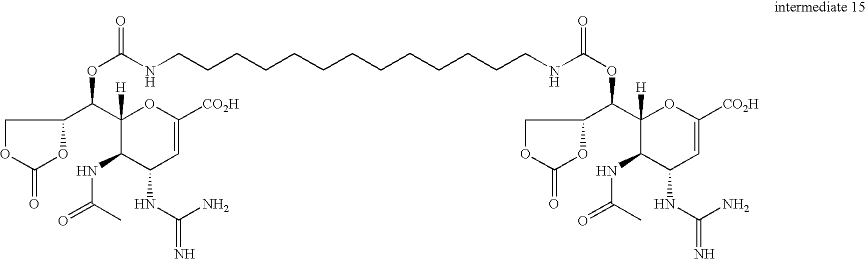

Intermediate 10 (0.16 g; 0.093 mmole) was dissolved in a 10:1 mixture of dichloromethane:anisole (6.3 ml) at room temperature. To this was added TFA (6.3 ml) and the resulting solution was stirred for 2.5 hours then evaporated in vacuo. Trituration of the residue with ether gave intermediate 15 as the di-TFA salt (92 mg; 82% yield). LC/MS (method A) showed (M+2H⁺)/2=492; T_{RET}=2.61 min.

Similarly prepared were the following:

| | starting | | | |
|---|---|---|---|---|
| n | material | product | (M + 2H⁺)/2 | T_{RET}(min) |
| 10 | intermediate 7 | intermediate 12 | 471 | 2.31 |
| 11 | intermediate 8 | intermediate 13 | 478 | 2.43 |
| 12 | intermediate 9 | intermediate 14 | 485 | 2.51 |
| 14 | intermediate 11 | intermediate 16 | 499 | 2.68 |

### Example 4

### n=13

### (2R,3R,4S)-3-(acetylamino)-2-{(1R,21R,22R)-21-((2R,3R,4S)-3-(acetylamino)-4-{[amino(imino)methyl]amino}-6-carboxy-3,4-dihydro-2H-pyran-2-yl)-1-[(1R)-1,2-dihydroxyethyl]-22,23-dihydroxy-3,19-dioxo-2,20-dioxa-4,18-diazatricos-1-yl}-4-{[amino(imino)methyl]amino}-3,4-dihydro-2H-pyran-6-carboxylic acid bis(trifluoroacetic acid salt)

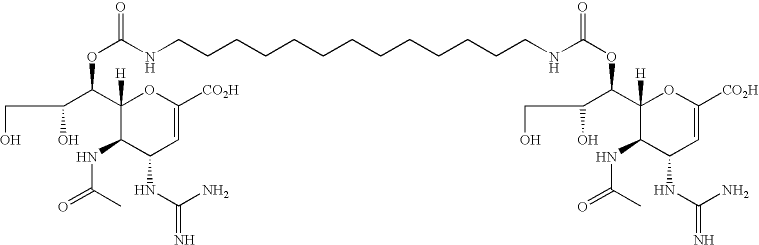

Intermediate 15 (92 mg; 0.076 mmole) was dissolved in a mixture of water (16ml) and methanol (16 ml). To this was added triethylamine (4 ml) and the solution was stirred for 1 hour. Volatile organics were removed in vacuo and the residue adjusted to pH 2 with TFA. Reverse phase preparative HPLC (method B) gave example 4 as the di-TFA salt (35.5 mg; 40% yield). LC/MS (method A) showed (M+2H⁺)/2=466; T_{RET}=2.45 min.

Elemental analysis: Found: C, 42.00; H, 5.79; N, 11.00%. Calc for tetrahydrate: C, 41.95; H, 6.18; N, 11.38%. NMR (D₂O)δ: 5.85 (2H, d, 2×CH); 4.85 (2H, dd, 2×CH); 4.46 (2H, dd, 2×CH); 4.34 (2H, dd, 2×CH); 4.05, 2H, t, 2×CH); 3.94 (2H, m, 2×CH); 3.58 (2H, dd, CH₂); 3.42 (2H, dd, CH₂); 2.95 (4H, m, 2×CH₂); 1.88 (6H, s, 2×CH₃); 1.38 (4H, br.m, 2×CH₂); 1.22-1.10 (18H, br.m, 9×CH₂) p.p.m.

### Example 4a

### Large Scale Preparation of Example 4

Intermediate 15 (2.8 g; 2.3 mmoles) was dissolved in water (50.4 ml). To this was added methanol (50.4 ml) followed by triethylamine (6.4 ml; 46 mmoles). The resulting solution was stirred at room temperature for 5 hours, the volume of the reaction mixture was reduced by ca 33% in vacuo at 35 degrees C. then the pH was adjusted to 2 with TFA (0.5 ml). The acidified solution was then injected onto a Prochom LC50 HPLC system comprising of a 20 cm×5 cm column packed with 7 micron Kromasil C8 packing material. The column was subjected to gradient elution:
Solvent A: water+1% TFA
Solvent B: 75% acetonitrile/water+1% TFA
Flow: 80 ml/min
Gradient: 0% B to 100% B/40 min

The appropriate fractions were combined and the acetonitrile was removed in vacuo at 35 degrees C. The aqueous residue was absorbed onto a 10 cm×22 mm column of Amberchrom CG-161 (PSDVB resin) and the column was washed with water then eluted with acetonitrile:MeOH:water 2:2:1 (500 ml). The solvent was removed in vacuo to yield a gum. The addition of isopropanol (20 ml) gave a solid which was dried to give the product as the zwitterion (1.68 g).

### Example 4b

### Crystallisation of Example 4

The zwitterion (100 mg; 0.1075 mmoles) was dissolved in water (35 ml). To this was added sodium bicarbonate (18.06 mg; 0.215 mmoles) and the resulting solution was freeze-dried to give a white solid. A sample (2 mg) of this solid was dissolved in water (0.8 ml) and evaporated to a syrupy oil. Dioxan (1 ml) was added and a white solid formed. The solid was allowed to settle and the supernatent was removed. Further dioxan (1 ml) was added and the supernatent was again removed. This process was repeated twice more and the solid obtained was dried in vacuo. Examination under polarised light showed crystallinity.

Examples E1, E2 and E3 were prepared using an analogous procedure to that of Example E4.

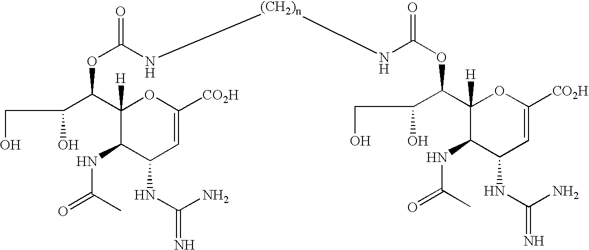

### Example 1

### n=11

### (2R,3R,4S)-3-(acetylamino)-2-{(1R,19R,20R)-19-((2R,3R,4S)-3-(acetylamino)-4-{[amino(imino)methyl]amino}-6-carboxy-3,4-dihydro-2H-pyran-2-yl)-1-[(1R)-1,2-dihydroxyethyl]-20,21-dihydroxy-3,17-dioxo-2,18-dioxa-4,16-diazahenicos-1-yl}-4-{[amino(imino)methyl]amino}-3,4-dihydro-2H-pyran-6-carboxylic acid bis(trifluoroacetic acid salt)

LC/MS (method A) showed (M+2H⁺)/2=452; T_{RET}=2.25 min.

### Example 2

### n=12

### (2R,3R,4S)-3-(acetylamino)-2-{(1R,20R,21R)-20-((2R,3R,4S)-3-(acetylamino)-4-{[amino(imino)methyl]amino}-6-carboxy-3,4-dihydro-2H-pyran-2-yl)-1-[(1R)-1,2-dihydroxyethyl]-21,22-dihydroxy-3,18-dioxo-2,19-dioxa-4,17-diazadocos-1-yl}-4-{[amino(imino)methyl]amino}-3,4-dihydro-2H-pyran-6-carboxylic acid bis(trifluoroacetic acid salt)

LC/MS (method A) showed (M+2H⁺)/2=459; T_{RET}=2.34 min.

### Example 3

### n=14

### (2R,3R,4S)-3-(acetylamino)-2-{(1R,22R,23R)-22-((2R,3R,4S)-3-(acetylamino)-4-{[amino(imino)methyl]amino}-6-carboxy-3,4-dihydro-2H-pyran-2-yl)-1-[(1R)-1,2-dihydroxyethyl]-23,24-dihydroxy-3,20-dioxo-2,21-dioxa-4,19-diazatetracos-1-yl}-4-{[amino(imino)methyl]amino}-3,4-dihydro-2H-pyran-6-carboxylic acid bis(trifluoroacetic acid salt)

LC/MS (method A) showed (M+2H⁺)/2=473; T_{RET}=2.50 min.

### Example 5

### Evaluation of the Compounds of Formula (I)—Inhibition of Influenza Virus Replication

Cytopathic effect (CPE) assays were performed essentially as described by Watanabe et al. (J. Virological Methods, 1994 48 257). MDCK cells were infected with a defined inoculum of virus (determined by experimentation to be the minimum sufficient to cause adequate CPE in 72 hours and to be susceptible to control compounds at concentrations considered to be consistent with published norms) in the presence serial dilutions of Compounds of the invention. Cultures were incubated for up to 72 hours at 37° C. in a 5% CO₂ atmosphere. The extent of CPE and hence viral replication was determined via metabolism of the viral dye 3-(4,5-dimethylthiazol-2-yl)-2,5-diphenyltetrazolium bromide (MTT) according to published methods (see for example, Watanabe et al., 1994). The compound concentration that inhibited CPE by 50% (ID₅₀) was calculated using a computer program for curve fitting. Influenza A/Sydney/5/97 and B/Harbin/7/95 viruses were assayed and the results are shown in Table 1. Comparable data for a specifically disclosed compound in WO 00/55149 and for compound A is also shown in Table 1.

TABLE 1

| | ID₅₀ μg/ml | ID₅₀ M | ID₅₀ μg/ml | ID₅₀ M |
|---|---|---|---|---|
| Description | A/Sydney/5/97+ | A/Sydney/5/97+ | B/Harbin/7/95 | B/Harbin/7/95 |
| Compound A | 0.023 +/− 0.024 | 69 | 0.013 +/− 0.011 | 39 |
| E1 | 0.0002 | 0.179 | 0.0001 | 0.09 |
| E1 | 0.0001 | 0.09 | 0.0001 | 0.09 |
| E2 | 0.0001, 0.0001 | 0.087 | 0.0001, 0.0001 | 0.087 |
| E3 | 0.0001 | 0.085 | 0.0003 | 0.26 |
| E4 | 0.0001 | 0.086 | 0.0001 | 0.086 |
| Compound | 0.0007, 0.0005 | 0.58, 0.75 | 0.007 +/− 0.01 | 5.8 |
| Number 8* | | | | |
| Compound | 0.057 | 66 | >0.1 | >115 |
| Number 10* | | | | |
| *As referenced in WO 00/55149 | | | | |
| +Data provided in WO 00/55149 related to the virus H₃N₂ isolate A/Victoria/3/75 rather than A H₃N₂ isolate A/Sydney/5/97. When comparing such data the person skilled in the art will appreciate that differences in | | | | |
| # antiviral potency are not uncommon for a given compound when analysed against several different viruses in vitro. For example, Woods et al (Antimicrob Agents Chemother 1993 37: 1473-9) have reported that Compound A exhibits a wide range of EC50 | | | | |
| # values (from 0.02 to 0.16 μM) in in vitro assays involving recent clinical isolates. Accordingly, compound 8 was found to be more potent in CPE assays involving the recent influenza A H₃N₂ isolate A/Sudney/5/97 than the earlier H₃N₂ | | | | |
| # isolate A/Victoria/3/75. | | | | |

antiviral potency are not uncommon for a given compound when analysed against several different viruses in vitro. For example, Woods et al (Antimicrob Agents Chemother 1993 37:1473-9) have reported that Compound A exhibits a wide range of EC50 values (from 0.02 to 0.16 μM) in in vitro assays involving recent clinical isolates. Accordingly, compound 8 was found to be more potent in CPE assays involving the recent influenza A H₃N₂ isolate A/Sydney/5/97 than the earlier H₃N₂ isolate A/Victoria/3/75.

Data provided in Table 1 demonstrate that the compounds E1-E5, in addition to being substantially more potent than the highly active compound A, are even more potent against A/Sydney/5/97 and substantially more potent against the recent influenza B isolate B/Harbin/7/95 than compounds 8 and 10 of WO 00/55149.

### Example 6

### Plaque Reduction Assay

Madin Darby Canine Kidney (MDCK) cells are seeded into six well tissue culture plates and grown to confluency via standard methods. Influenza viruses are diluted in a minimal volume of phosphate buffered saline supplemented with 0.2% bovine serum albumin to yield an estimated titre of 50-100 plaque forming units (pfu) per well. After adsorption to the MDCK cells for one hour at 37° C. in a 5% CO₂ atmosphere the viral inocula is aspirated and replaced with viral growth media (minimal Eagle's media supplemented with BSA, trypsin and insulin/transferrin/selenium at optimal concentrations) containing sufficient agar or agarose (generally 1-2%) to cause the media to gel at room temperature and at 37° C. in a 5% CO₂ atmosphere until plaques develop (generally 2-4 days). Plaques can be visualised with a suitable stain (e.g. 0.4% crystal violet in formal saline) before counting. Antiviral potency is expressed as the concentration of test article which reduces plaque numbers by 50% of the untreated control value (EC₅₀).

| | EC₅₀ ng/ml | | | | | |
|---|---|---|---|---|---|---|
| | PRA | | | | | |
| Example | A/WSN* | A/Vic* | A/Syd* | A/New* | A/Pan* | A/Bay* |
| Compound A | 56, >100 | 5.5 +/− 8.2 | 2.4 | 0.27, 0.23 | 2.7, 3 | 35 |
| 3 | | 0.0023 | 0.000429 | | | |
| 2 | | 0.06, 0.2 | <0.0001 | | | |
| 4 | <0.0001 | <0.001, <0.01, 0.2 | <0.0001 | 0.043 | <0.00001 | |
| 5 | <0.0001 | <0.001, 0.02, 0.3 | 0.032 | <0.0001 | 0.032 | <0.0001 |
| Amantadine | | 220 | | 11 | 157 | |
| Oseltamivir | | 0.11 | | 0.23 | 0.3 | |

*A/WSN/33 BVLV09 (H1N1)
A/Victoria/3/75 BVLV017 (H3N2)
A/Sydney/5/97 BVLV015 (H3N2)
A/New Caledonia/20/99 BVLV008 (H1N1)
A/Panama/2007/99 BVLV008 (H3N2)
A/Bayern/7/95 BVL006 (H1N1)

| | EC₅₀ ng/ml | | | |
|---|---|---|---|---|
| | PRA | | | |
| Example | B/Vic* | B/Harb* | B/HongK* | B/Yam* |
| Compound A | 3, 20 | 0.19 | 21 +/− 6 | 0.2, 3.1 |
| 3 | 0.009, 0.01 | | <0.0001 | <0.0001, <0.0001 |
| 2 | 0.04, 0.05 | | | <0.0001 |
| 4 | 0.01, 0.1 | | 0.06 | <0.0001 |
| 5 | 0.05, 0.1 | | 0.37 | <0.0001 |
| Amantadine | | | >10000 | 2061 |
| Oseltamivir | | | 32 | 0.7 |

*B/Victoria/1/67
B/Hong Kong/5/72 BVLV012
B/Harbin/7/95 BVLV008
B/Yamanashi/166/98 BVLV007

### Example 7

### Assessment of Long Duration of Action

Rodents are anaesthetised and dosed with compound of interest by the intra-tracheal route at a dose volume of 0.8 ml/kg. The rodent is then held in the vertical position until full recovery is achieved. At different time points, for example, 2, 8, 24 and 48 hours post-dose, levels of compound in the lung tissue are assessed by analytical methods. Any analytical method suitable for detection of this type of compound may be used. The time at which levels of compound fall below the sensitivity of the analytical techniques identified will determine the residency time of the compound in lung tissue.

### Example 8

### Alternative Assessment of Long Duration of Action and Efficacy

The protocol for infecting mice has been described previously (1-4). Mildly anaesthetised mice are inoculated into the external nares with influenza virus. Treatment procedure and regimen. A single dose of compound is administered at a defined time point up to 10 days prior to infection, preferably 4-7 days prior to infection, or following infection, preferably immediately following infection and up to 48 hours post infection. In most experiments, a non-lethal strain of influenza is used, and efficacy is assessed by reductions in lung virus titre. For mice given compound prior to infection, lungs are removed post infection either on a single day, or on days following infection, preferably days 1-4 post infection. Homogenised lung samples are assayed for virus using established methods, and the titres of viral load estimated and compared to titres of virus in lungs of untreated mice.

In those experiments where a mouse-adapted lethal strain of influenza is used, efficacy is assessed by an increase in survival rate and/or numbers of survivors, as compared to untreated mice.

Antibacterial Agents

### EXAMPLES 9 TO 11

### Example 9

### Aminoglycoside Dimers

Aminoglycoside intermediates 1 and 2 were prepared as shown below using adaptations of procedures previously published by Michael, K. et al., Bioorg. Med. Chem. 1999, 7 1361-1371.

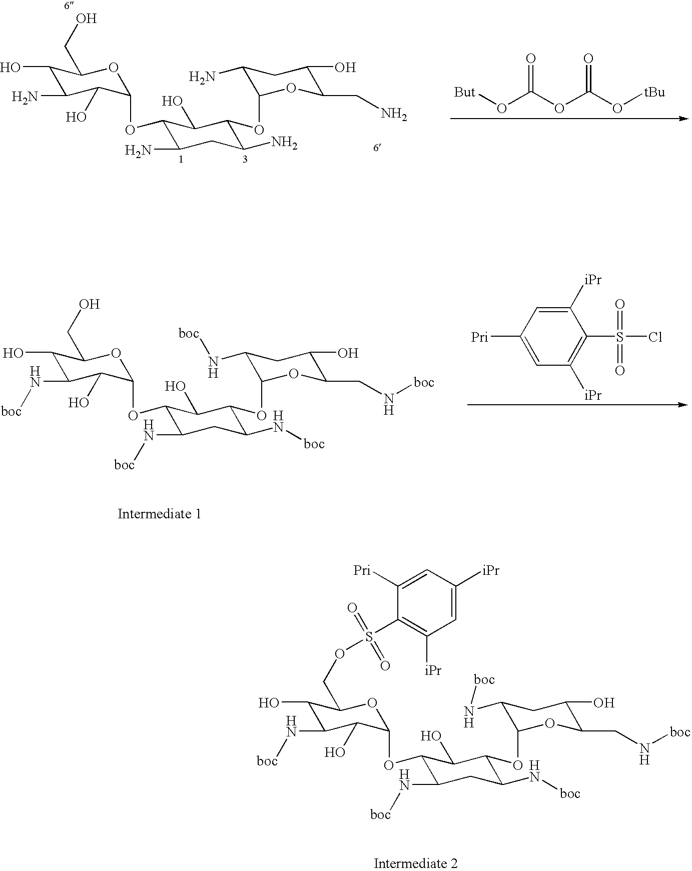

Intermediates 3 and 4, were prepared as shown below using adaptations of the previously described method of Van Schepdael, A., et al., J. Med. Chem. 1991, 34, 1468-1475. 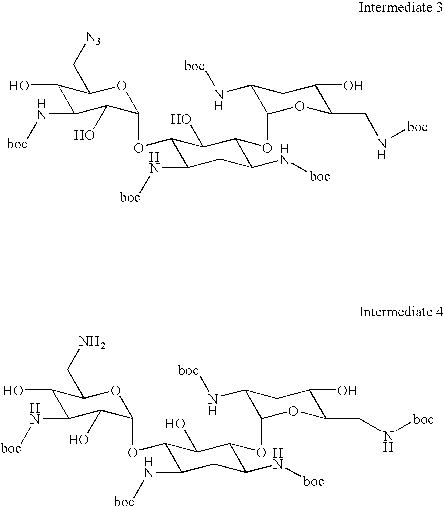
General Method for the Preparation of Intermediates 5, 6a-c, 7

A solution of Intermediate 2 (123 mg, 0.1 mmole) in ethanol (10 ml) was treated with either piperazine, 1,2-ethylenediamine, 1,3-propanediamine,1,4-butanediamine or, hydrazine hydrate (55% aqueous solution) as appropriate (1 g) and the solution was heated at reflux for 16 h. The ethanol was removed under vacuum, the residue triturated with water (4 ml) and the product collected by filtration. The filter cake was washed with water (2×5 ml) and air-dried. The identities of the products were verified by m/s and used without further purification.

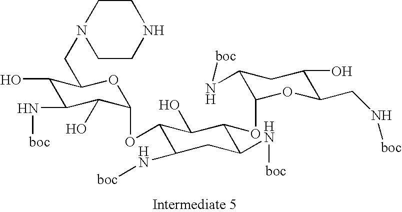

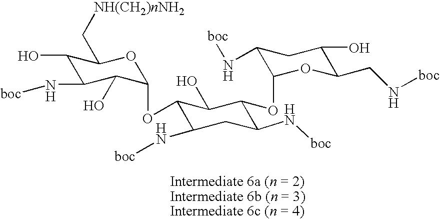

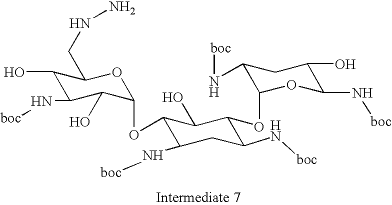

| Compound | Calculated MW | Observed m/z |
|---|---|---|
| Intermediate 3 | 992 | 993[M + H]⁺ |
| | | 1015 [M + Na]⁺ |
| Intermediate 4 | 996 | 967 [M + H]⁺ |
| Intermediate 5 | 1035 | 1036 [M + H]⁺ |
| Intermediate 6a | 1009 | 1010 [M + H]⁺ |
| | | 506 [M + 2H]²⁺ |
| Intermediate 6b | 1023 | 1024 [M + H}⁺; |
| | | 513 [M + 2H]²⁺ |
| Intermediate 6c | 1037 | 1039 [M + H]⁺ |
| | | 520 [M + 2H]²⁺ |
| Intermediate 7 | 981 | 982 [M +H]⁺ |

Dimerisation Reactions Conditions
Method A: Urea and Urethane Dimers Prepared Using the 6″ Position as a Point of Attachment

A solution of a suitable boc-protected tobramycin derivative (Intermediates 1, 5, 6a, 6b, 6c) (0.03 mmole) in anhydrous DMF (0.5 ml) was treated with an appropriate diisocyanate (0.01 mmole). The mixture was then stirred and heated at 70 degrees Celsius for 24 h under nitrogen. An aliquot was removed and analyzed by ES-MS and, if necessary, the conversion could be improved by further addition of diisocyanate up to a maximum of 0.5 equivalents to 1 equivalent of amine, and heating and stirring continued for 24 h. Upon complete conversion, the DMF was removed in vacuo and the residue dissolved in trifluoroacetic acid (2 ml) at room temperature. After 5 min. the solution was evaporated to dryness with minimal heating to afford the crude dimer. The product was isolated by chromatography, conditions for which are provided in Table 3.

Method B: Amide Dimers

To a solution of a boc-protected tobramycin derivative (Intermediates 5, 6a, 6b, 6c) (24.4 μmole) in DMF (1 mL) was added the solution of the appropriate dicarboxylic acid (1.25 μmole), BOP (24.4 μmole) in DMF (1 mL). After 5 min, diisopropylethylamine (51.2 μmole) was added and the mixture was allowed to stir overnight at room temperature under argon. Upon completion of the reaction, the DMF was removed in vacuo and the residue was treated with CH₂Cl₂/TFA (1:1, 1 mL) for 4 h at room temperature then concentrated. The remaining residue was redissolved in H₂O (5 mL) then freeze-dried to provide the crude product. The product was isolated by chromatography, conditions for which are provided in Table 3.

Method C: Alkylamine Dimers

A suspension of a boc-protected tobramycin derivative (Intermediates 5, 6a, 6b, 6c) (0.03 mmole), anhydrous potassium carbonate (20 mg) in anhydrous DMF (0.5 ml) was treated with the appropriate dibromide (0.01 mmole). The mixture was stirred and heated at 70 degrees Celsius for 24 h under nitrogen. An aliquot was then removed and analyzed by ES-MS and, if necessary, the conversion could be improved by further addition of dibromide up to a maximum of 0.5 equivalents to 1 equivalent of amine, and heating and stirring continued for 24 h. Upon complete conversion, the DMF was removed in vacuo and the residue dissolved in trifluoroacetic acid (2 ml) at room temperature. After 5 min. the solution was evaporated to dryness with minimal heating to afford the crude dimer. The product was isolated by chromatography, conditions for which are provided in Table 3.

General Procedure for the Preparation of Aminoglycoside Dimers Using the 6′ Position as a Point of Attachment

Method D: Urea Dimers

A solution of tobramycin (0.5 mmole) in DMF (1 ml)/water (1 ml) was cooled in an ice bath. The appropriate diisocyanate was added and the mixture was stirred at room temperature for 18 h. The mixture was filtered and the filtrate evaporated to dryness. The product was isolated by chromatography, conditions for which are provided in Table 3.

Method E: General Procedure for the Preparation for Tobramycin 6″ Triazole Dimers

The 6″ azide (intermediate 3) (0.055 mmole) and the appropriate diacetylene (0.025 mmole) were suspended in a 1:1 mixture of water and tert-butyl alcohol (2 ml). Sodium ascorbate (1M soln. in water, 0.055 ml), followed by a solution of copper sulfate (0.01 mmole) in water (0.030 ml) were added. The heterogeneous mixture was stirred vigorously for 1 hour then a further 1 ml of tert-butyl alcohol was added. Vigorous stirring was continued for 120 hours and then further copper sulphate pentahydrate (3 mg), and sodium ascorbate (0.5 ml, 1M solution), and the diacetylene (2 mg) were added. After a further 120 h of vigorous stirring, the reaction mixture was evaporated under vacuum to low volume and extracted with dichloromethane (3×10 ml) and ethyl acetate (3×10 ml). The solutions were dried over sodium sulfate and concentrated then redissolved in trifluoroacetic acid (3 ml) at room temp and allowed to stand for 5 minutes. The trifluoroacetic acid was removed under vacuum with the minimum of heating and the residual trifluoroacetic acid salts purified by chromatography.

Method F: Preparation of Imine Dimers Using the 6′ Position as a Point of Attachment

A solution of tobramycin (0.1 g, 0.214 mmol) and appropriate dialdehyde (0.076 mmol) in DMF (3 ml) was stirred overnight. The solvent was removed under reduced pressure to obtain crude diimine and the compound was purified by RP-HPLC.

Method G: Heterodimerisation

A stirred solution of Tobramycin (0.1 mmole) and intermediate 5 (0.025 mmole) in dimethylformamide/water 1:1 (1 ml) was cooled in ice. The diisocyanate (0.1 mmole) was added and the reaction was allowed to warm to room temperature and stirred for 18 h. The reaction was diluted with water (5 ml), stirred for 1 h, then filtered. The aqueous filtrate was evaporated to dryness under vacuum and the residue suspended in dichloromethane (1 ml) then triethylsilane (100 μl) was added, followed by trifluroacetic acid (1 ml). The mixture was stirred at room temperature for 4 h, evaporated to dryness and purified by HPLC.

Method H: Heterodimerisation

A stirred solution of Tobramycin (0.1 mmole) and Amikacin (0.05 mmole) in water (2 ml) was cooled in ice. The diisocyanate (0.1 mmole dissolved in 200 μL DMF) was added and the reaction was allowed to warm to room temperature and stirred for 48 h. The reaction was treated with water (2 ml), stirred for 1 h, then filtered. The amikacin-tobramycin heterodimer was found to reside in the aqueous filtrate.

Method I: Imine Formation

Tobramycin (210 μmole) and the dialdehyde (90 μmole) were dissolved in DMF and the solution stirred under argon for 18 hours. The product was identified and isolated by LCMS.

Method J: Reductive Amination

Tobramycin (210 μmole) and the dialdehyde (90 μmole) were dissolved in methanol then treated with sodium triacetoxyborohydride (45 mg) and the solution stirred under argon for 18 hours. The product was identified and isolated by LCMS.

TABLE 2

| | | Amino- | | |
|---|---|---|---|---|
| | | glycoside | General | |
| Compound | Linker reagent | Intermediate | Method | Compound Structure |
| 1 | 1,12- Diisocyanatododecane | 1 | A | 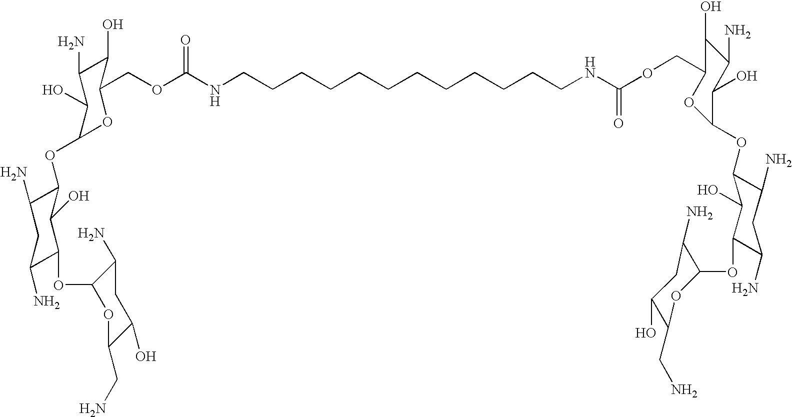 |
| 2 | NA (Prepared by treatment of Intermediate 4 with TFA) | NA | NA | 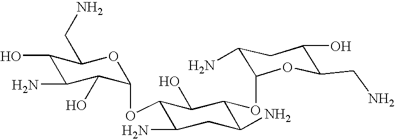 |
| 3 | Undecanedioic acid | Tobramycin | B | 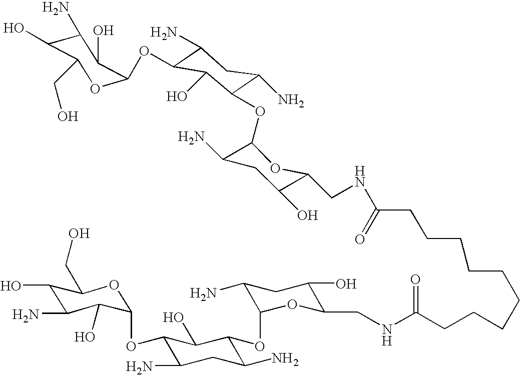 |
| 4 | 4,4′- Methylenebis(phenyl isocyanate) | 1 | A | 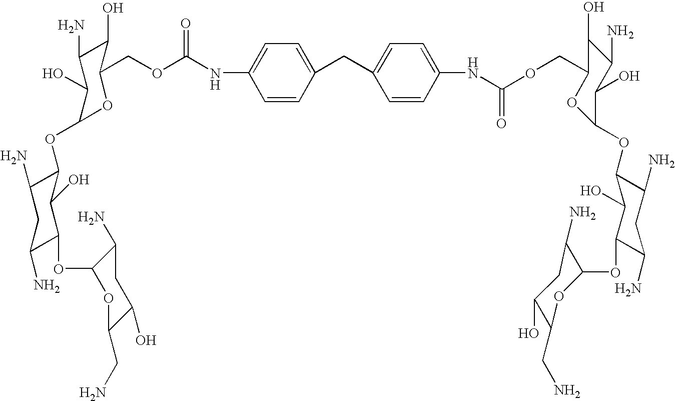 |
| 5 | NA (Prepared by Intermediate 5 with TFA) | NA | NA | 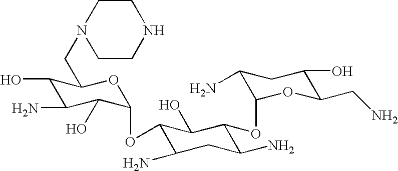 |
| 7 | Pentadecanedioic acid | 5 | B | 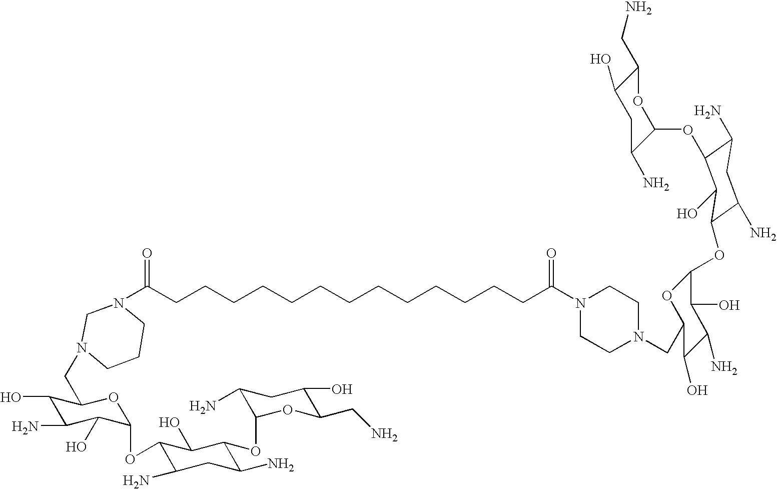 |
| 8 | Diphenic acid | 5 | B | 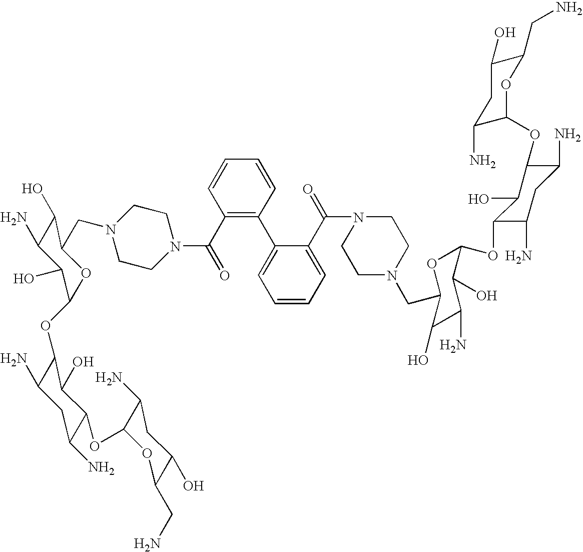 |
| 10 | Terephthalic acid | 6b | B | 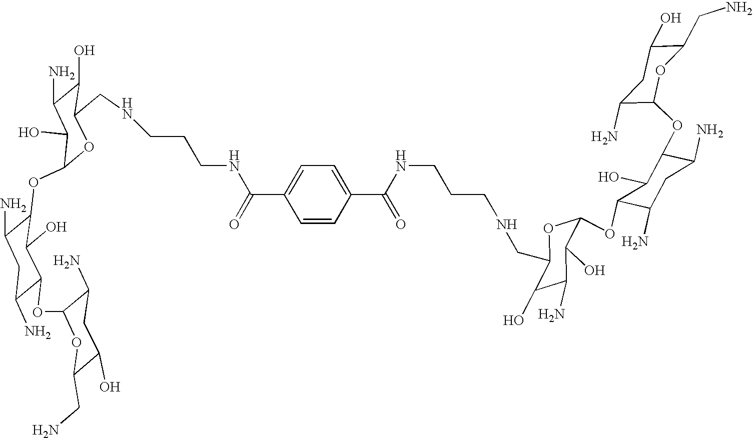 |
| 11 | Pyridine-3,5- dicarboxylic acid | 5 | B | 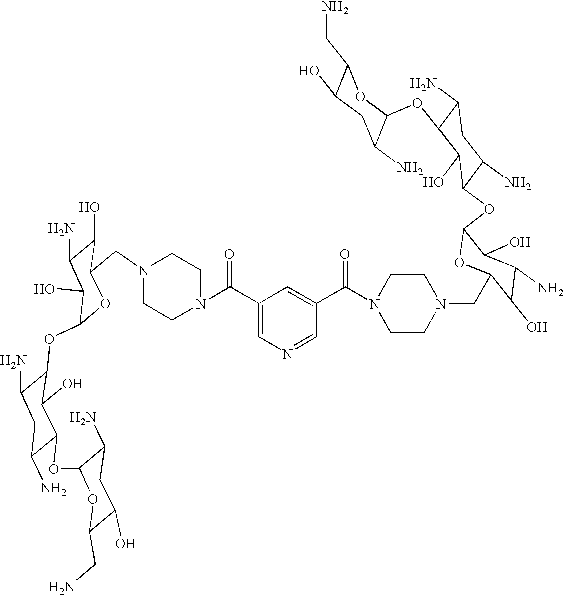 |
| 12 | Hexadecanedioic acid | 5 | B | 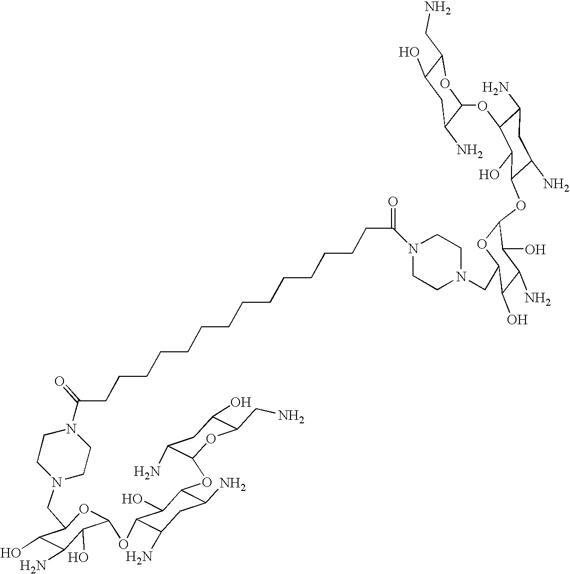 |
| 13 | 4,4′- Methylenebis(phenyl isocyanate | 4 | A | 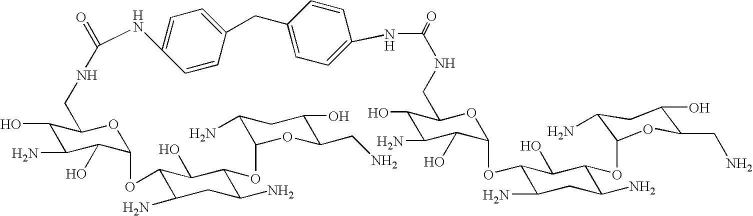 |
| 15 | 4,4′- Methylenebis(phenyl isocyanate) | 5 | A | 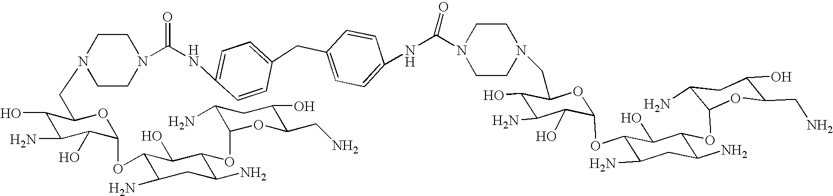 |
| 16 | 2,3,5-Trimethyl-1,3- phenyldiisocyanate | 5 | A | 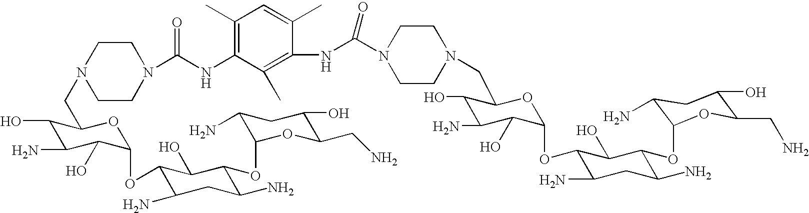 |
| 17 | 1,3- Bisbromomethyl- benzene | 5 | C | 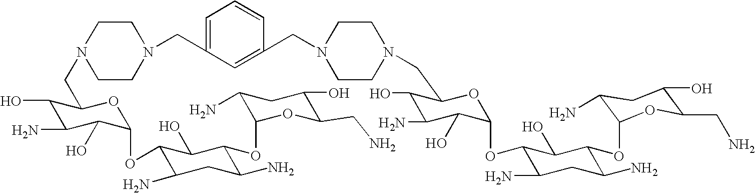 |
| 18 | 1,12- Diisocyanatododecane | Tobramycin | D | 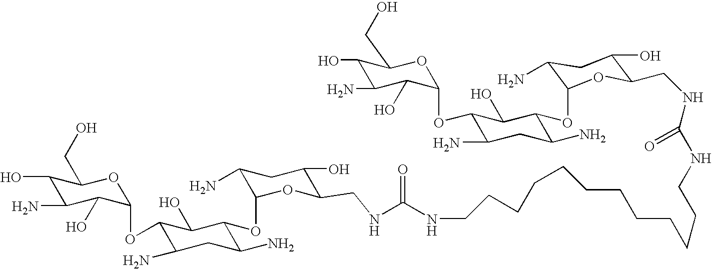 |
| 19 | 1,4- Phenyldiisocyanate | 5 | A | 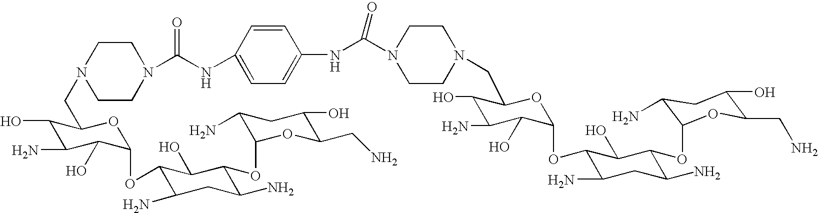 |
| 20 | 1.12- Dibromododecane | 5 | C | 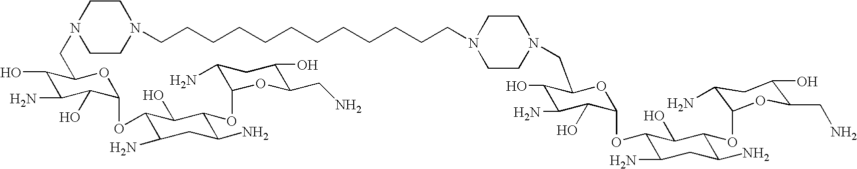 |
| 21 | 1,12- diisocyanatododecane | 4 | A | 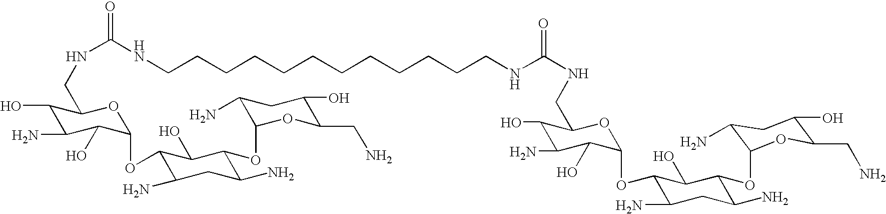 |
| 22 | 4,4′- Methylenebis(phenyl isocyanate) | 6c | A | 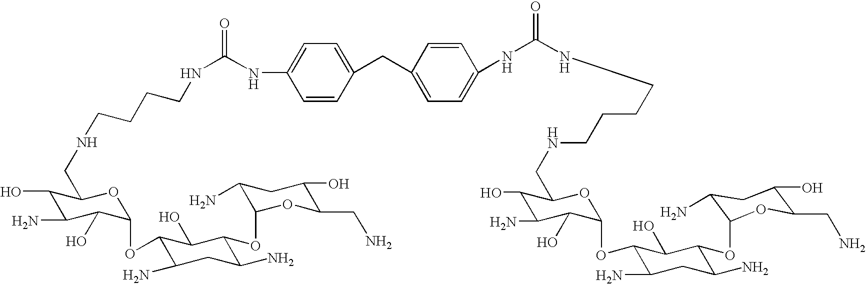 |
| 23 | Diphenic acid | 6b | B | 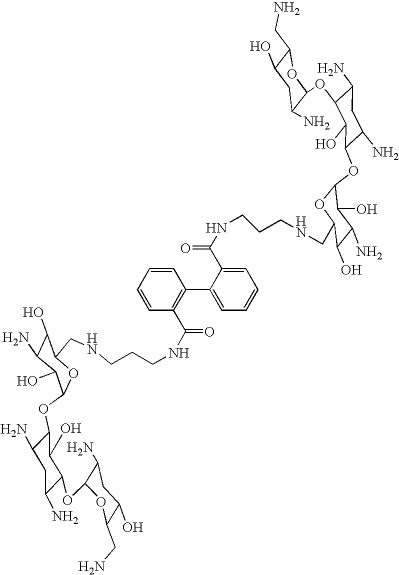 |
| 24 | Diphenic acid | 6a | B | 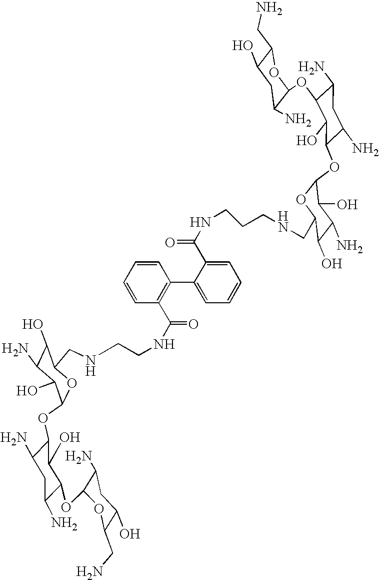 |
| 25 | Succinic acid | 5 | B | 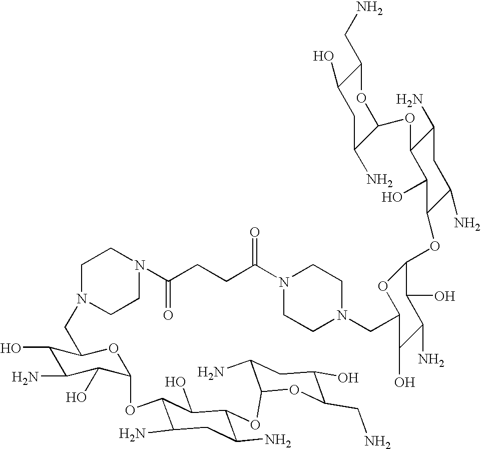 |
| 26 | Isophthalic acid | 6b | B | 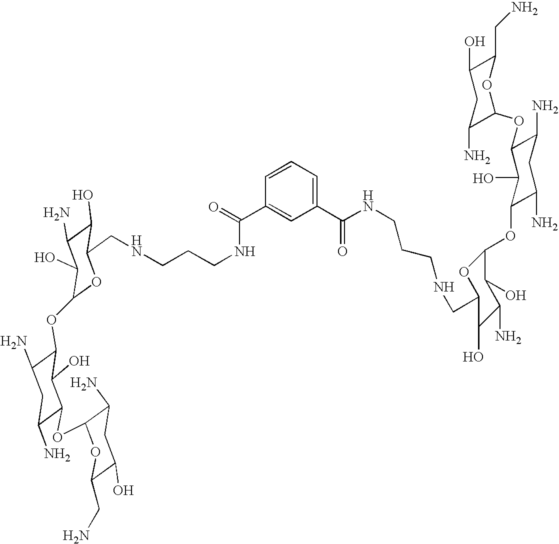 |
| 27 | Isophthalic acid | 6a | B | 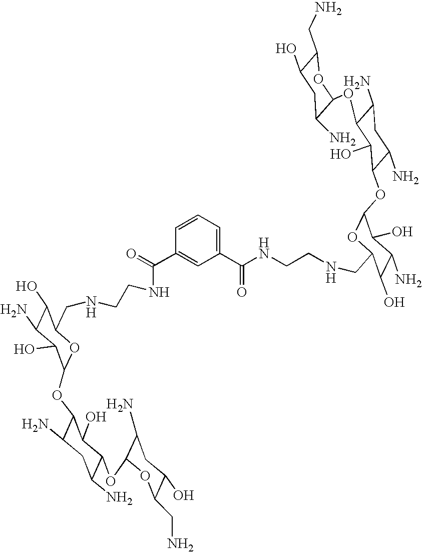 |
| 28 | Terephthalic acid | 6a | B | 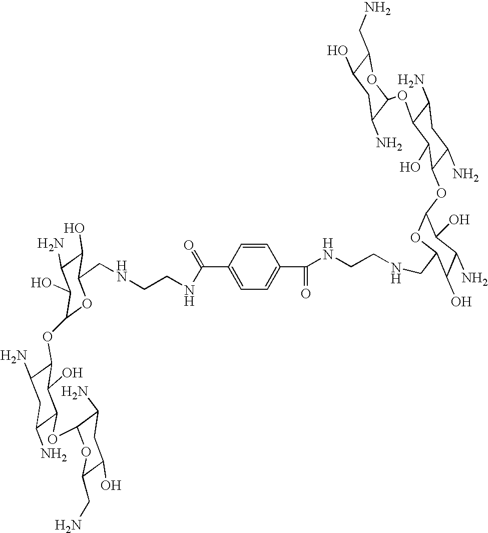 |
| 29 | 2,3,5- Trimethyl-1,3- phenyldiisocyanate | 6c | A | 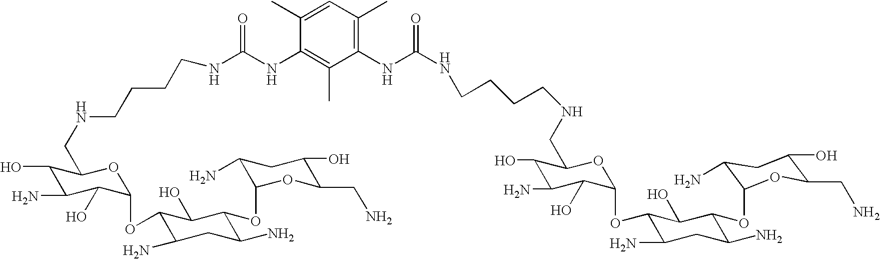 |
| 30 | 1,15- Hexadecadiyne | 3 | E | 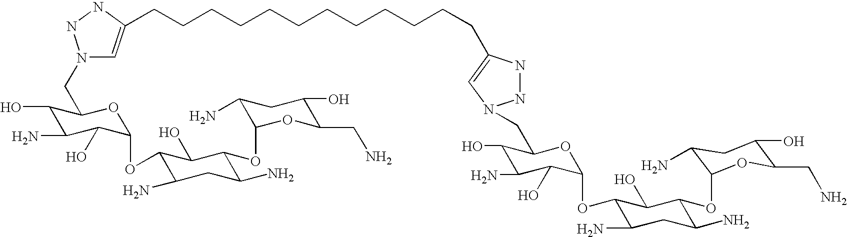 |
| 32 | 1,12- Diisocyanatododecane | Tobramycin/ Intermed. 5 | G | 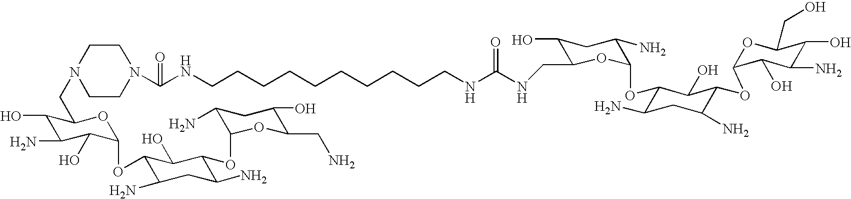 |
| 33 | 1,6- Diisocyanatohexane | 5 | A | 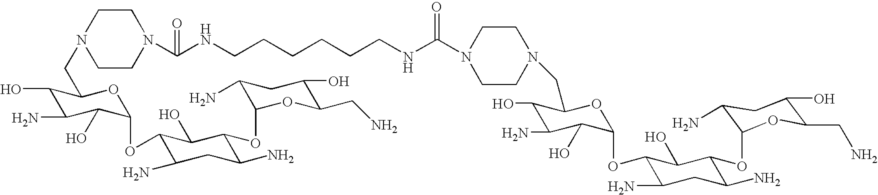 |
| 34 | 1,12- Diisocyanatododecane | Amikacin/ Tobramycin | G | 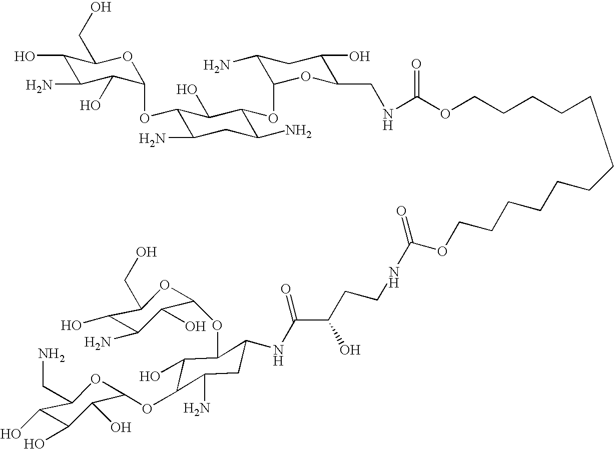 |

Characterisation Data for Aminoglycoside Analogues

| | Method RED | Method GREEN | Method BLUE |
|---|---|---|---|
| Column support and | Phenomenex 10μ C8 | Alltima C18 (5u × 150 mm × 4.6 mm) | Phenomenex 10μ C8 |
| dimensions | SiO₂ (25 cm × 2.12 cm) | | SiO₂ (25 cm × 2.12 cm) |
| Flow rate | 10 ml/min | 1.2 ml/min | 10 ml/min |
| Solvent A | 0.1% TFA/H₂O | 0.025% Formic acid in | 0.1% TFA/H₂O |
| | | H₂O | |
| Solvent B | 0.06% TFA/CH₃CN | 0.025% Formic acid in | 0.06% TFA/CH₃CN |
| | | CH₃CN | |

TABLE 3

| | | | | GRADIENT/HPLC |
|---|---|---|---|---|
| | | | HPLC | Method |
| | | | Retention | Initial % B-Final |
| Compound ID | Calculated MW | Observed m/z | time (min) | % B_Duration |
| 1 | 1186 | 1187.2[M + H]⁺ | 14.8 | Blue: |
| | | 594.3[M + 2H]²⁺ | | 15-80_28 min |
| 2 | 466 | 467[M + H]⁺ | 18.4 | Red: |
| | | | | 0-20_20 min |
| 3 | 1128 | 1129.6[M + H]⁺ | 24.2 | Blue: |
| | | 565.4[M + 2H]²⁺ | | 0-40_28_28 min |
| 4 | 1184 | 1185.5[M + H]⁺ | 28.5 | Blue: |
| | | 593.3[M + 2H]²⁺ | | 5-25_28 min |
| 5 | 535 | 536.4[M + H]⁺ | 18.7 | Red: |
| | | 269[M + 2H]²⁺ | | 0-20_20 min |
| 7 | 1307 | 1308.2[M + H]⁺ | 20.6 | Red: |
| | | 654.8[M + 2H]²⁺ | | 20-70_20 min |
| 8 | 1276 | 1278.1[M + H]⁺ | 8.9 | Blue: |
| | | 639.7[M + 2H]²⁺ | | 10-20_28 mins |
| 10 | 1176 | 589.7[M + 2H]²⁺ | 18.3 | Red: |
| | | | | 10-50_20 min |
| 11 | 1201 | 602.1[M + 2H]²⁺ | 24.3 | Red: |
| | | | | 10-50_20 min |
| 12 | 1320 | 1322.1[M + H]⁺ | 31.0 | Red: |
| | | 661.8[M + 2H]²⁺ | | 20-40_20 mins |
| 13 | 1182 | 1183[M + H]⁺; | 21 | Blue: |
| | | 592[M + 2H]²⁺ | | 5-30_28 mins |
| 15 | 1321 | 1322[M + H]⁺; | 9.35 | Blue: |
| | | 661[M + 2H]²⁺ | | 5-35_28 mins |
| 16 | 1273 | 1274[M + H]⁺ | 33.12 | Red: |
| | | 637[M + 2H]²⁺ | | 5-50_20 min |
| 17 | 1173 | 1174[M + H]⁺; | 32.92 | Red: |
| | | 587[M + 2H]²⁺ | | 5-50_20 min |
| 18 | 1186 | 1187[M + H]⁺; | 31.78 | Red: |
| | | 594[M + 2H]²⁺ | | 15-50 20 min |
| 19 | 1230 | 1231[M + H]⁺ | 16.57 | Red: |
| | | 616[M + 2H]²⁺ | | 15-20_20 min |
| 20 | 1236 | 619[M + 2H]²⁺ | 22.99 | Red: |
| | | | | 15-50_20 min |
| 21 | 1184 | 593[M + 2H]²⁺ | 34.89 | Red: |
| | | | | 5-50_20 min |
| 22 | 1325 | 663[M + 2H]²⁺ | | |
| 23 | 1253 | 1254.3[M + H]⁺ | 19.5 | Blue: |
| | | 627.8[M + 2H]²⁺ | | 10-25_28 min |
| 24 | 1225 | 613.4[M + 2H]²⁺ | 19.87 | Red: |
| | | | | 10-50_20 min |
| 25 | 1153 | 577.7[M + 2H]²⁺ | 23.15 | Red: |
| | | | | 10-50_20 min |
| 26 | 1177 | 1177.6[M + H]⁺ | 10.5 | Blue: |
| | | 589.5[M + 2H]² | | 10-30_28 min |
| 27 | 1149 | 1149.8[M + H]⁺ | 10 | Blue: |
| | | 575.4[M + 2H]² | | 10-25_28 mis |
| 28 | 1149 | 1149.9[M + H]⁺ | 10 | Blue: |
| | | 575.4[M + 2H]² | | 10-40_28 min |
| 29 | 1277 | 1278.1[M + H]⁺ | 18.5 | Blue: |
| | | 639.8[M + 2H]² | | 10-30_28 min |
| 30 | 1203 | 1203.9[M + H]⁺ | 22 | Blue: |
| | | 602.8[M + 2H] | | 5-50%_28 min |
| 32 | 1254 | 628[M + 2H]²⁺ | 4.07 | Green: |
| | | | | 5-60%_10 min |
| 33 | 1239 | 1240[M + H]⁺ | 1.3 | Green: |
| | | 621[M + 2H]²⁺ | | 5-60%_10 min |
| 34 | 1306 | 1307[M + H]⁺ | ND | ND |
| 35 | 1032 | 1033[M + H]⁺ | ND | ND |
| 36 | 1036 | 1037[M + H]⁺ | 1.6 | Green: |
| | | 519[M + 2H]²⁺ | | 5-60%_10 min |

### Example 10

### Antibiotic Activities of Aminoglycoside Dimers

Antibiotic activities were determined using a standard Kirby-Bauer* test and are reported as diameters (in mm) of zones of inhibition for test strains. All compounds were spotted at 30 μg/disk.

| | | | Pseudomonas | Staphylococcus |
|---|---|---|---|---|
| | Escherichia coli | Enterococcus faecalis | aeuruginosa | aureus |
| Compound | ATTCC 25922 | ATCC 29212 | ATCC 27853 | ATCC 29213 |
| Tobramycin | 18.4 | 15.7 | 24.9 | 26.7 |
| Amikacin | 18.5 | 6.7 | 18.4 | 18.2 |
| Neomycin | 14.4 | 6.7 | 7.1 | 16.8 |
| Kanamycin A | 18.2 | 6.7 | 6.2 | 19.1 |
| 1 | 7.0 | 6.0 | 6.3 | 8.9 |
| 2 | 6.3 | 6.3 | 12.8 | 18.2 |
| 3 | 6.2 | 6.0 | 6.0 | 11.7 |
| 4 | 7.9 | 6.0 | 9.2 | 9.3 |
| 5 | 10.5 | 7.4 | 13.8 | 18.6 |
| 7 | 6.5 | 6.2 | 8.9 | ND |
| 8 | 11.4 | 6.2 | 12.1 | ND |
| 10 | ND | ND | ND | ND |
| 11 | ND | ND | ND | ND |
| 12 | ND | ND | ND | ND |
| 13 | 7.1 | 6.5 | 8.9 | 8.6 |
| 15 | 7.7 | 6.2 | 9.0 | ND |
| 16 | 6.5 | 6.2 | 10.1 | ND |
| 17 | 6.5 | 6.2 | 8.5 | ND |
| 18 | 7.6 | 6.2 | 10.4 | ND |
| 19 | 12.1 | 6.2 | 12.8 | ND |
| 20 | 7.8 | 6.2 | 7.5 | ND |
| 21 | ND | ND | ND | ND |
| 22 | ND | ND | 6.0 | ND |
| 23 | ND | ND | ND | ND |
| 24 | ND | ND | 8.9 | ND |
| 25 | ND | ND | 6.0 | ND |
| 26 | ND | ND | ND | ND |
| 27 | ND | ND | ND | ND |
| 28 | ND | ND | ND | ND |
| 29 | ND | ND | ND | ND |
| 30 | ND | ND | ND | ND |
| 32 | ND | ND | ND | ND |
| 33 | ND | ND | ND | ND |

*
1. Bauer, A. W., Kirby, W. M. M., Sherris, J. C., Turck, M. Antibiotic susceptibility testing by standardised single disk method. Am. J. Clin. Pathol. 1966; 45: 493-496.
2. Performance Standards for Antimicrobial Disk Susceptibility Tests; Approved Standard-Seventh Edition, January 2000, M2-A7, Vol. 20 No. 1 (Replaces M2-A6, Vol. 17 No. 1). NCCLS.

### Example 11

### Ciprofloxacin Dimers

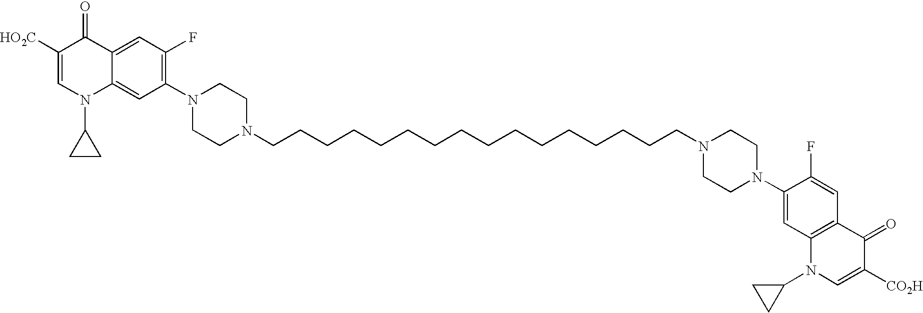

Ciprofloxacin hydrochloride (58 mg, 157 μmole) and hexadecanedial (20 mg, 79 μmole) were dissolved in 1,2-dichloroethane (1 mL) and treated with triethylamine (44 μL, 314 μmole). The solution was placed under an argon atmosphere and treated with sodium triacetoxyborohydride (47 mg, 22 μmole). After 96 hours stirring at ambient temperatures, the reaction was quenched by addition of 1 mL satd. aqueous sodium bicarbonate then 1 mL water and diluted with chloroform (5 mL). The layers were separated and the aqueous layer further extracted with chloroform (3×5 mL). The combined organic extracts were dried (Na₂SO₄), filtered and concentrated to provide the crude product. Purification was accomplished by precipitation of the product from an organic solvent such as chloroform.

HPLC. T_{RET} 8.65 min. Conditions. Column: Phenomenex Luna C8 (100 mm×4.60 mm); Detection: 214 nm; Flow: 1.5 ml/min. Eluents: Solvent A: acetonitrile+0.06% TFA; Solvent B: water+0.1% TFA. Gradient: 5-100% A/6 min, 100% A/2 min, 100-5% A/1 min.

ESMS. Calculated for C₅₀H₆₆F₂N₆O₂ 884. Found m/z 885 [M+H]+

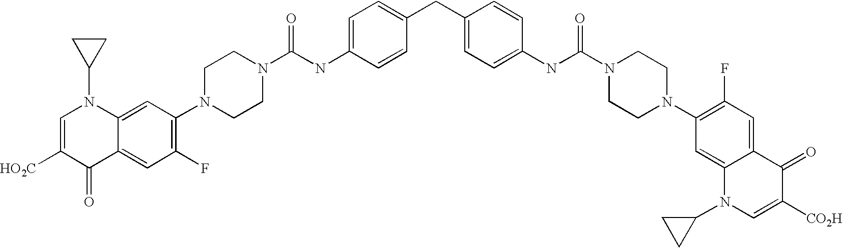

A suspension of ciprofloxacin hydrochloride (100 mg, 270 μmole) and potassium carbonate (138 mg, 1.00 mmole) in DMF (3 mL) was treated with 4,4′-methylenebis(phenyl isocyanate) (34 mg, 130 μmole). The suspension was placed under an argon atmosphere and stirred for 22 hours at ambient temperatures.

ESMS. Calculated for C₄₉H₄₆F₂N₈O₈ 912. Found m/z 951 [M+K]+

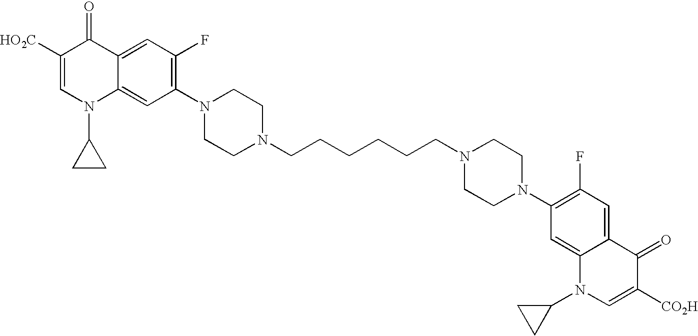

A suspension of ciprofloxacin hydrochloride (100 mg, 270 μmole) and potassium carbonate (182 mg, 1.32 mmole) in DMF (2 mL) was treated with 1,6-dibromohexane (321 μL, 130 μmole). The suspension was placed under an argon atmosphere and stirred for 22 hours at ambient temperatures.

ESMS. Calculated for C₄₀H₄₆F₂N₆O₆ 744. Found m/z 783 [M+K]+

### Example 12

### Assessment of Long Duration of Action

Rodents were anaesthetised with Ketamine/Domitor mixture according to standard procedures and dosed with compound of interest by the intra-nasal route at a dose volume of approximately 3.0 ml/kg. The rodent is held in the vertical position during dosing of 30 μL per nostril. At different time points, for example, 2, 8, 24, 48 and 168 hours post-dose, levels of compound in the lung tissue are assessed by analytical methods. Any analytical method suitable for detection of this type of compound may be used. The time at which levels of compound fall below the sensitivity of the analytical techniques identified will determine the residency time of the compound in lung tissue.

Homo- and heterodimeric compounds disclosed herein were found to be retained in the mouse lung for longer periods than related monomeric compounds. Selected data are shown below.

| | nmole retained per |
|---|---|
| | gram of mouse lung |
| Compound | after 7 days |
| Tobramycin | 2.1 |
| 5 | 0.8 |
| 8 | 4.6 |
| 16 | 2.8 |
| 18 | 6.6 |

### REFERENCES

1. Katja Michael, Hai Wang and Yitzhak Tor, 1999. Enhanced RNA binding of dimerized aminoglycosides. Bioorg. Med. Chem Lett., 7, 1361-1371.
2. Yuan-Ping Pang, Polly Quiram, Tanya Jelacic, Feng Hong, and Stephen Brimijoin, 1996. Highly Potent, Selective, and Low Cost Bis-tetrahydroaminacrine Inhibitors of Acetylcholinesterase. J. Biol. Chem. 271: 23646-23649.
3. Ryan, D. M., J. Ticehurst, M. H. Dempsey, and C. R. Penn, 1994. Inhibition of influenza virus replication in mice by GG167 (4-guanidino-2,4-dideoxy-2,3-dehydro-N-acetylneuraminic acid) is consistent with extracellular activity of viral neuraminidase (sialidase). Antimicrob. Agents and Chemother. 38 (10):2270-2275.
4. von Itzstein M., W. -Y. Wu, G. B. Kok, M. S. Pegg, J. C. Dyason, B. Jin, T. V. Phan, M. L. Smythe, H. F. White, S. W. Oliver, P. M. Colman, J. N. Varghese, D. M. Ryan, J. M. Woods, R. C. Bethell, V. J. Hogham, J. M. Cameron, and C. R. Penn. 1993. Rational design of potent sialidase-based inhibitors of influenza virus replication. Nature (London) 363:418-423.
5. Woods, J. M., R. C. Bethell, J. A. V. Coates, N. Healey, S. A. Hiscox, B. A. Pearson, D. M. Ryan, J. Ticehurst, J. Tilling, S. A. Walcott, and C. R. Penn. 1993. 4-Guanidino-2,4-dideoxy-2,3-dehydro-N-acetylneuraminic acid is a highly effective inhibitor both of the sialidase (neuraminidase) and of growth of a wide range of influenza A and B viruses in vitro. Antimicrob. Agents Chemother. 37:1473-1479.
6. Robert J Fenton, Peter J Morley, Ian J Owens, David Gower, Simon Parry, Lee Crossman and Tony Wong (1999). Chemoprophylaxis of influenza A virus infections, with single doses of zanamivir, demonstrates that zanamivir is cleared slowly from the respiratory tract. Antimicrob. Agents and Chemother. 43, 11, 2642-2647.

1. A compound of general formula (I):
X-L-Y (I)
in which
X and Y are pharmaceutically active moieties which are the same or different and selected from antiviral agents excluding inhibitors of influenza neuraminidase; aminoglycosides selected from tobramycin, kanamycin, amikacin and neomycin; antifungal agents; and antiparasitic agents; and
L is a linker which is an optionally substituted saturated or unsaturated straight chain, branched, and/or cyclic hydrocarbon radical having a backbone of at least 11 atoms with the proviso that the linker does not contain one or more disulphide bonds,
or a pharmaceutically acceptable derivative or salt thereof.

2. The compound according to claim 1, wherein the aminoglycoside is tobramycin and/or amikacin.

3. The compound according to claim 1, wherein the antifungal agent is amphotericin β or an azole.

4. The compound according to claim 1, wherein the antiparasitic agent is an aspartic proteinase.

5. The compound according to claim 1, wherein the hydrocarbon radical is selected from the group consisting of alkyl, alkenyl, alkynyl, cycloalkyl, cycloalkenyl, heterocyclyl and aryl.

6. The compound according to claim 1, wherein the linker includes one or more N, 0, S and/or functional groups.

7. The compound according to claim 6, wherein the functional group is an amide, amine, carbonyl arid/or carboxy.

8. The compound according to claim 1, wherein the linker is a straight chain or branched alkyl having 12 to 16 carbon atoms.

9. The compound according to claim 8, wherein the alkyl has 13 or 14 carbon atoms.

10. The compound according to claim 1, wherein the linker comprises an optionally substituted phenyl, optionally substituted biphenyl or an optionally substituted heterocyclyl.

11. The compound according to claim 1, wherein said compound has a modification at one or more of the carboxyl, hydroxyl, amino, carbonyl or ether functional groups.

12. The compound according to claim 1, wherein the modification is an ether, alkyl ester, an aryl ester or an acetyl ester.

13. A pharmaceutical formulation comprising a compound of formula (I) as defined in claim 1 or a pharmaceutically acceptable salt or derivative thereof, together with one or more pharmaceutically acceptable carriers.

14. The pharmaceutical formulation according to claim 13, which further comprises one or more other therapeutic and/or prophylactic ingredients.

15. The pharmaceutical formulation according to claim 14, wherein the other therapeutic and/or prophylactic ingredient is an antiinfective agent.

16. The pharmaceutical formulation according to claim 15, wherein the antiinfective agent is an antiviral or antibacterial agent.

17. The pharmaceutical formulation according to claim 16, wherein the antibacterial or antiviral agents are those used to treat respiratory infections.

18. The pharmaceutical formulation according to claim 17, wherein the agent is tobramycin and/or amikacin.

19. An inhaler which comprises a compound of formula (I) or a pharmaceutically acceptable salt or derivative thereof, according to claim 1 or a formulation comprising a compound of formula (I) or a pharmaceutically acceptable salt or derivative thereof, together with one or more pharmaceutically acceptable carriers.

20. The inhaler according to claim 19, wherein said inhaler is adapted for oral administration as a free-flow powder.

21. The inhaler according to claim 19, wherein said inhaler is a metered dose aerosol inhaler.

22. A method for the prevention or treatment of a microbial infection comprising the step of administering to a subject in need thereof of an effective amount of a compound of formula (I) as defined in claim 1.

23. The method according to claim 22, wherein the microbial infection is a viral, bacterial, fungal, parasitic, yeast or protozoal infection.

24. The method according to claim 23, wherein the bacterial infection is a Gram Negative or Gram positive infection.

25. The method according to claim 24, wherein the bacterial infection is associated with the respiratory tract, urinary tract or GI tract or a systemic infection caused by enteric bacteria.

26. The method according to claim 22, wherein administering is to the respiratory tract by inhalation, insufflation or intranasally or a combination thereof.

27-29. (canceled)

30. A method for the detection of a microbial infection which comprises the step of contacting the compound of formula (I) as defined in claim 1 with a sample suspected of containing a microorganism.

31. A method for the preparation of the compound of formula (I) as defined in claim 1 which comprises the steps of:
(a) optionally protecting moieties X and Y;
(b) reacting the optionally protected moieties X and Y with the optionally protected linker L; and
(c) if necessary, deprotecting the protected compound of formula (I).
